(19) United States
(12) Patent Application Publication
(10) Pub. No.: US 2026/0256811 A1
(43) Pub. Date: Sep. 3, 2026

(54) Humoral Immune Activators for the Treatment of Humoral Immunosuppressed Pathologies, Cancer and Infectious Disease and Humoral Immune Suppressors for the Treatment of Inflammatory Diseases

(71) Applicant: Navrogen, Inc., Glen Mills, PA (US)

(72) Inventors: Nicholas C. Nicolaides, Glen Mills, PA (US); James Bradford Kline, Morgantown, PA (US); Luigi Grasso, Bryn Mawr, PA (US)

(73) Assignee: Navrogen, Inc., Glen Mills, PA (US)

(21) Appl. No.: 18/867,520

(22) PCT Filed: Jun. 15, 2023
(86) PCT No.: PCT/US2023/068472

§ 371 Date: Nov. 20, 2024

Related U.S. Application Data
(60) Provisional application No. 63/352,420, filed on Jun. 15, 2022.

(51) Int. Cl. A61K 31/58; A61K 9/00; A61K 31/575; A61K 47/68; C07J 43/00; G01N 33/50; G01N 33/68

(52) U.S. Cl. CPC A61K 31/58; A61K 9/0053; A61K 31/575; A61K 47/6803; C07J 43/003; G01N 33/502; G01N 33/6854; G01N 2333/525; G01N 2333/535; G01N 2333/545; G01N 2333/57; G01N 2333/70535; G01N 2333/96436; G01N 2500/20

(57) ABSTRACT

Compositions, methods, and kits of small molecular weight agents can bind and stimulate the Fc-γ-activating receptors to enhance humoral immune-response and immune-effector cell response by improving immune-effector cell and/or antibody-based efficacy against pathogenic microbes as well as virally infected and dysregulated cells. These can be used to treat infectious disease, cancer and other humoral immunosuppressed diseases. In addition, non-stimulatory analogs that can block Fc-γ-activating receptors can be used to suppress immune-effector cell activation and downstream mediators of inflammation.

### TECHNICAL FIELD OF THE INVENTION

The invention relates to the area of humoral immunity and immune-effector cell regulation. In particular, it relates to compositions, methods, and kits of small molecular weight agents that bind and stimulate Fc-Y-activating receptors, thereby enhancing humoral immune-responses. Stimulation of the Fc-γ-activating receptors improves immune-effector cell mediated and antibody mediated responses against pathogenic microbes, virally infected cells and dysregulated cells. The small molecular weight agents can be used to treat infectious diseases, cancers, and other humoral immunosuppressed diseases. In addition, non-stimulatory analogs that can bind and block CD16a activation can be used in treating inflammatory diseases mediated by CD16a expressing cells.

### BACKGROUND OF THE INVENTION

Humoral immunity is a major mechanism by which vertebrate host organisms surveil and defend against infectious pathogens and dysregulated host cells. In cancer therapy, monoclonal antibodies (mAbs) that can target cancer-specific cell surface antigens have been successfully developed and commercially approved. Several of these mAbs have been reported to exert their tumor-killing effects through humoral-mediated antibody dependent cellular cytotoxicity (ADCC) and/or antibody dependent cellular phagocytosis (ADCP) via engagement with Fc-γ-activating receptors such as CD16a (FCGRIIIA), CD32a (FCGRIIA) and CD64 (FCGRIA) on immune-effector cells (NK cells, macrophages, neutrophils, monocytes, dendritic cells, etc.), in addition to complement dependent cytotoxicity (CDC) (DiLillo D J, Ravetech J V, Cancer Immunol Res 3:704-713, 2015; Ruck T, et al. Int J Mol Sci. 16:16414-16439, 2015; Pelaia C, et al. Biomed Res Int 4839230: 1-9, 2018; VanDerMeid K R, et al. Cancer Immunol Res 6:1150-1160, 2018). Recently, several investigators have reported that tumors produce factors that can suppress humoral immune pathways that in turn suppress the tumor-killing effects of mAbs by ADCC, ADCP and/or CDC mechanisms (Vergote I, et al. J Clin Oncol 34:2271-2278; Kline J B, et al. J Clin Oncol 5:15, 2018; Wang W et al. Cytogenet Genome Res 152:169-179, 2017; Kline J B et al. Eur J Immunol. 48:1872-1882, 2018). ADCC, ADCP and CDC humoral immune responses are governed by the coordination of antibodies and cell surface antigen engagement that together position the antibody on the antigen epitope at a certain proximity to the cell surface. In cases where this positioning is optimal, cell surface bound antibodies may engage with Fc-Y-activating receptors on Natural Killer (NK) or dendritic/myeloid/monocytic cells (any cell that participates in ADCC or ADCP is referred to here as an “immune-effector cell”) to initiate ADCC or ADCP as well as engage with the Clq complement initiating protein to cause death of antibody-bound cells via the classical complement pathway (Reuschenbach M, et al. Cancer Immunol Immunother 58:1535-1544, 2009). These effects have been observed for several therapeutic antibodies such as but not limited to rituximab, trastuzumab, cetuximab, pertuzumab, alemtuzumab and daratumumab (Zhou X, et al. Oncologist 13:954-966, 2008; Hsu Y F, et al. Mol Cancer 9:1-8, 2010; Spiridon C I, et al. Clin Cancer Res 8:1720-1730, 2002; Luo C, et al. Sci Rep 7: 46347, 2017; Sanchez L, et al. J Hematol Oncol 9:51-59, 2016). Similarly, humoral effects have been observed within a patient's own immune response to dysregulated cells and in reaction to vaccines. Antibodies with anti-proliferative as well as immune-mediated killing activities have been observed in patients with indolent disease. (Staff C, et al. J Clin Immunol 32:855-865; Branden S, et al. Cancer Res 63:7995-8005, 2003). Yet, while many patients afflicted with cancer and viral disease have been found to produce autoantibodies to tumor or viral antigens, often their presence is not sufficient for eradicating diseased cells, which is likely due to overall low self-immune-inducing antibody levels, suboptimal epitope binding, immune-effector cell exhaustion and/or humoral immunosuppressive mechanisms by the pathogen, virally infected or dysregulated cell (Bi J and Tian Z. Frontiers Immunol 8:1-10, 2017).

In cancer (also referred to here as dysregulated cells), the tumor-produced protein MUC16/CA125 has been found to suppress humoral immune responses via direct binding to a subset of IgG1, IgG3 and IgM type antibodies that in turn perturb the Fc region making it less effective for their engagement with Fc-γ-activating receptors FCGR2A (also referred to as FCGRIIA and CD32a) and/or FCGR3A (also referred to as FCGRIIIA and CD16a) on immune-effector cells or engagement with the Clq protein for CDC (Pantankar M S, et. al. Gyncol Oncol 99:704-713, 2005; Kline J B, et al. OncoTarget 8:52045-52060, 2017; Kline J B, et al. J. Clin. Oncol. 5:15, 2018; Wang W, et al. Cytogenet Genome Res 152:169-179, 2017; Kline J B, et al. Eur J Immunol. 48:1872-1882, 2018; Vergote I, et al. J Clin Oncol 34:2271-2278, 2016; Nicolaides N C, et al. Cancer Biol Ther 13:1-22, 2018; Prochazka V, et al. Int J Hematol 96:58-64, 2012; Grasso L. Oncology Letters 23:2, 2022). This effect is not observed on the high affinity FCGR1 (also referred to as CD64) Fc-γ-receptor (K_{D}=0.5 nM), suggesting that this effect is due in part to the low affinity binding of CD16a allotypes 158F (1059 nM) and 158V (415 nM) as well as CD32a allotypes 131R (218 nM) and 131H (39 nM) (Kline J B, et al. J. Clin. Oncol. 5:15, 2018; Nordstrom J L, et al. Breast Cancer Res 13: R123, 2011).

Much of the above findings around humoral immunosuppression in cancer have pointed to the importance of NK and myeloid cell activation and antibody binding for effective killing. Several reports have shown removal of NK cells reduces the therapeutic efficacy of antibody-mediated anti-cancer cell response (Waldhauer I, Steinle A. Oncogene 27:5932-5943, 2008). Based on these observations, several approaches to utilize NK cells for cancer immunotherapy are being pursued in experimental models and clinical trial settings (Vivier E, et al. Nat Rev Immunol 12:239-252, 2012; Shimasaki N, et. al. Nat Rev Drug Dis 19:200-218, 2020). These approaches aim to utilize ADCC as well as antibody independent cellular cytotoxicity (AICC) killing mechanisms by NK cells against host target cells lacking major histocompatibility complex class 1 (MHC1) protein due to viral infection or dysregulation (Bachanova V and Miller J S. Crit Rev Oncol 19:133-141, 2014; Paul S and Lal G. Front Immunol 8:1124, 2017). Moreover, NK cell therapy is also being pursued for the treatment of virally infected patients to eradicate virally infected host cells that are downstream sources for viral reactivation (Traylen C M, et. al. Future Virol 6:451-463, 2011, Larkin J, et. al. J Interferon Cytokine Res 26:12, 2006). A major drawback to utilizing NK cells directly or by antibody-mediated therapeutic response is the inability to retain sufficient numbers of activated cells that can attack virally infected or dysregulated cells after administration to the patient. Several attempts to improve NK cell activation via pretreatment or concomitant treatment with cytokine agonists such as IL-15 or stimulatory agents that block NK cell negative regulatory pathways such as NKG2A have had mixed outcomes (Knudsen K M et al. Exp Opinion Biol Ther 20:7 705-709, 2020; Miller J S et al. Nature Med 28:392-400, 2022; Creelan B C and Antonia S J. Nature Rev Clin Oncol 16:277-278, 2019). These suboptimal activities may be due to either off-target effects leading to host toxicity or limited stimulation in vivo (Guo, J et al. Cell Res 31:1190-1198, 2021). In light of these drawbacks, a need exists for the identification of agents that may preferentially or specifically activate and maintain NK and other immune-effector cell killing activity toward viral-infected or dysregulated cells.

CD16a expressing cells have also been found to be associated and directly involved in inflammatory diseases (Cooper D L, et al. PLOS 7: e28918, 2012). Several association studies testing cellular infiltrates in rheumatoid arthritis have found a correlation of CD16a expressing cells and severity of disease (Robinson J I, et al. Ann Rheum Dis 69:1054-1057, 2010). Moreover, experimental animal models of inflammatory disease have shown a direct impact of CD16a activity and inflammatory disease progression (Ji H, et al. Immunity 16:157-168, 2002; Diaz D S, et al. Eur J Immunol 32:2915-2922, 2002). Based on these findings, the development of agents that can block CD16a activation may be useful as therapeutics for CD16a-associated inflammatory diseases.

### SUMMARY OF THE INVENTION

The present invention provides naturally occurring and synthetic small molecular weight agents that enhance IgG antibody binding to the CD16a-158F Fc receptor (FcR) (SEQ ID NO: 1) and to the CD16a-158V Fc receptor (SEQ ID NO: 2), as well as stimulate CD16a FcR on immune-effector cells for cellular activation and downstream target cell killing. The small molecular weight agents invention are related to lanosta-8,24-dien-3β-ol (Compound 1a) and 9β-19-cyclo-24-lanosten-3β-ol (Compound 2a) of the class of tetracyclic triterpenoids. They are capable of enhancing CD16a-158F FcR and its high affinity allotype CD16a-158V FcR binding to IgG1 antibodies as well as of activating the CD16a pathway in NK and other immune-effector cells. This class of therapeutics can be used for the treatment of infectious diseases, cancer, and other humoral immunosuppressed diseases via CD16a activation of immune-effector cells (see also Nes W D and Heftmann E. Nat Prod 44:377-400, 1981). Alternatively, the use of these scaffolds can be altered at certain carbons to bind CD16a and block its unwanted activation in inflammatory diseases.

The cholestane-based lanosta-8,24-dien-3β-ol (Compound 1a and 1b); 9β-19-cyclo-24-lanosten-3β-ol (Compound 2a and 2b); bisnoralcohol-based Compounds 3, 4a and 4b; as well as analogs comprising their three cyclohexyl (A, B, C) and cyclopentyl (D) ring backbone structure as scaffolds, can be used as described for altering CD16a to enhance affinity binding to IgG-type antibodies and/or CD16a receptor activation in the presence or absence of IgG while other analogs may be used to block CD16a binding to IgG and blocking its activation. Small molecular weight agents based on the following structures have been analyzed and tested:

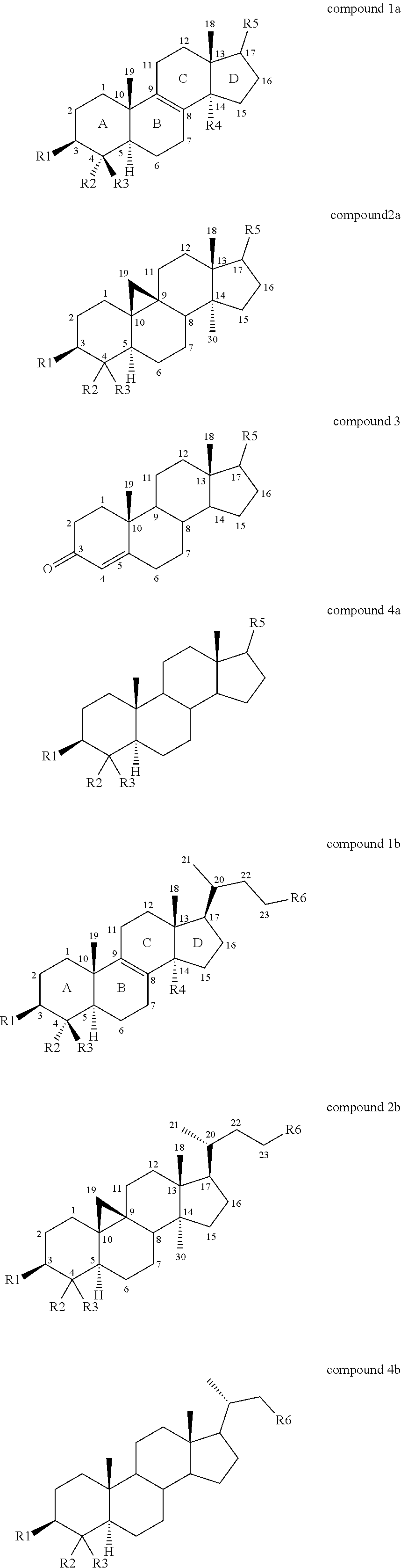

wherein R¹ is hydroxyl (OH) or formyl (CH═O);
wherein R² and R³ are methyl (CH₃);
wherein R⁴ is a methyl or 14α-difluoro-methyl (CHF₂);
wherein R⁵ and R⁶ are independently aldehyde (CH₃CHO); amine (NH₂); dimethylamine ((CH₃)₂NH); piperidine ((CH₂)₅NH); isopropyl amine ((CH₃)₂CHNH₂); morpholine (O(CH₂CH₂)₂NH); carboxylic acid (C(═O) OH); sulfonamide (CH₃SO₂NH₂); acetamide (CH₃CONH₂) or (C₂H₅NO); alcohol (OH); ketone (CH₃C(O) CH₃), isopropyl methyl ketone (CH₃COCH(CH₃)₂) or methyl-iso-butyl-ketone ((CH₃)₂CHCH₂COCH₃); methyl (CH₃); 2-methylheptane 2,3-diol (CH₃CH(CH₂)CH(OH)C(OH)(CH₃)₂; a 2-methylheptane (CH₃CH(CH₂)₃CH(CH₃)₂; a pegylated amine comprising 2 to 12 PEG (polyethylene glycol) units; a PEG5 amine; and a biotinylated amine, wherein a biotin moiety is linked to an amine moiety by PEG 2-12.

An example of a biotinylated PEG2 amine that can be used as R6 is:

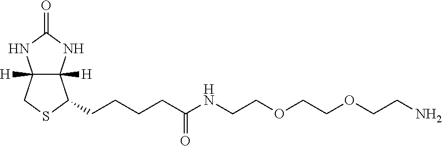 [SMILES: NCCOCCOCCNC(=O)CCCC[C@@H]1SC[C@@H]2NC(=O)N[C@@H]21]

An example of a PEG5 amine being used as R6 is:

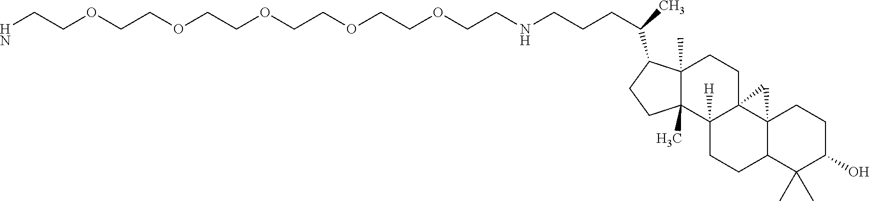

Another aspect of the invention employs the above compounds, as well as certain other compounds, as effective binders to CD16a FcR and stimulators of immune-effector cells expressing such receptors to enhance therapeutic effects against pathogens as well as virally infected and dysregulated cells, including malignant cells, in patients.

The present invention also includes therapeutic pharmaceutical compositions for activating immune-effector cells in vitro and in vivo in mammals, including humans. The pharmaceutical compositions comprise an effective amount of a Compound 1a, 1b, 2a, 2b, 3, 4a, or 4b.

Several of the above compounds are preferred for reasons such as increased ease of synthesis and/or greater efficacy. Preferred compounds include lanosterol14α-difluoro-methyl, lanosterol 24-aldehyde, lanosterol 24-amine, lanosterol 24-dimethylamine, lanosterol 24-piperidine, lanosterol 24-isopropylamine, lanosterol 24-morpholine, lanosterol 24 carboxylic acid, lanosterol 24-sulfonamide, lanosterol 24-acetamide, lanosterol 24-alcohol, lanosterol 24-PEG (2-12), lanosterol 24-biotin, lanosterol 24-PEG5-biotin, lanosterol 24-PEG5-lanosterol, lanosterol 24-PEG5-cycloartenol, lanosterol 24-isopropyl methyl ketone, lanosterol 24-methyl-iso-butyl-ketone, lanosterol 24-methyl, dihydrolanosterol, and 24, 25 dihydroxylanosterol.

Other preferred small molecular weight agents which may be used are: cycloartenol 24-aldehyde, cycloartenol 24-amine, cycloartenol 24-dimethylamine, cycloartenol 24-piperidine, cycloartenol 24-isopropylamine, cycloartenol 24-morpholine, cycloartenol 24 carboxylic acid, cycloartenol 24-sulfonamide, cycloartenol 24-acetamide, cycloartenol 24-alcohol, cycloartenol 24-PEG (2-12), cycloartenol 24-biotin, cycloartenol 24-PEG5-biotin, cycloartenol 24-PEG5-lanosterol, cycloartenol 24-PEG5-cycloartenol, cycloartenol 24-isopropyl methyl ketone, cycloartenol 24-methyl-iso-butyl-ketone, cycloartenol 24-methyl.

Other embodiments include the formulation of the above-described small molecular weight agents and analogs employing parenteral and non-parenteral pharmaceutical formulations. Such pharmaceutical formulations may include but are not limited to alpha-tocophenol, cetyl alcohol, cetearyl alcohol, corn oil mono-di glyceride, cremophor EL, enteric acrylic resins, ethanol, ethyl oleate, glycerol, isopropyl palmitate, lauryl lactate, liposomes, mono/diglycerides of caprylic/capric acids, oleic acid, PEG, phosphate buffered saline, polysorbate 80, propylene glycol, sorbitan monocleate, sulfonamides, and trehalose.

Another embodiment of the invention employs CD16a in a molecular binding assay, measuring binding affinity of CD16a to full length IgG antibody or IgG Fc fragments in the presence of Compound 1a, 1b, 2a, 2b, 3, 4a, or 4b via any ELISA-based or affinity kinetic-based methods such as surface plasmon resonance (SPR), fluidics-based binding such as Octet™ assays, or other kinetic binding analytical platforms.

Another aspect of the invention is the application of a biotinylated Compound 1a, 1b, 2a, 2b, 3, 4a, or 4b to monitor binding of said biotinylated compound to CD16a domains. This may be used for delineation of critical binding domains, for improving antibody binding affinity, for improving CD16a FcR activation, and/or for determining biological processing of activated FcR in CD16a expressing cells.

Another aspect of the invention is the use of Compound 1a, 1b, 2a, 2b, 3, 4a, or 4b to enhance CD16a to IgG binding in molecular assays. This can be used to facilitate the screening for inhibitors of CD16a to IgG Fc binding. The use of such compounds enhances the binding signal, which may facilitate screening for such inhibitors.

Another aspect of the invention is a kit for enhancing CD16a binding to IgG Fc domain for in vitro screening. The kit comprises Compound 1a, 1b, 2a, 2b, 3, 4a, or 4b, which can be used in CD16a to IgG Fc binding assays to enhance binding signal. The enhanced binding signal may be used to test for inhibitors of CD16a to IgG Fc binding. Any method known by those skilled in the art for screening of protein-protein antagonists may be used.

Yet another aspect of the invention is the conjugation of Compound 1a, 1b, 2a, 2b, 3, 4a, or 4b to an antibody to enhance the antibody's binding to CD16a and to improve antibody-mediated therapeutic responses.

Yet another aspect of the invention is the administration of Compound 1a, 1b, 2a, 2b, 3, 4a, or 4b to a patient with infectious disease or cancer, with or without standard-of-care therapy. The compound enhances the patient's humoral immune response (i.e., activation of CD16a expressing immune-effector cells) thereby improving clinical outcome. Compounds may be administered orally, dermally and/or by injection.

Other embodiments of the invention are to employ analogs of compounds 1a, 1b, 2a, 2b, 3, 4a, and/or 4b that bind CD16a and block its activation in immune-effector cells to suppress inflammation.

These and other aspects of the invention which will be apparent to those skilled in the art upon reading the specification, provide the art with methods, compositions, and kits for use in enhancing CD16a-expressing immune-effector cells, including but not limited to NK and myeloid cells. These can be used to improve patient response against pathogen or viral infection, as well as against dysregulated cells, including cancer. Compounds of the invention can be used as a single agent or in combination with other therapies, including those that are antibody based. The antibody may optionally be conjugated to one or more of the compounds. The compounds may improve antibody-mediated humoral immune responses in diseases associated with immunosuppression, including cancer and non-oncologic diseases in which antibody therapeutics are used to stimulate humoral-mediated immune responses. A second aspect of the invention is the use of compounds that block CD16a activation to suppress immune-effector cell activation that in turn supports unwanted inflammation for the treatment of inflammatory diseases.

### BRIEF DESCRIPTION OF THE DRAWINGS

FIG. 1A-1B. Compound 1 and Compound 2 specifically enhance human (FIG. 1A) and rodent (FIG. 1B) CD16a Fc receptor binding to IgG antibody Fc domain. These data identify rodents as relevant species to test Compounds 1 and 2 as well as analogs for enhancing CD16a-expressing immune-effector cell activation for in vivo therapeutic activity. Experiments represent a minimum of triplicate wells. P values were generated using the Student's T-test.

FIG. 2. Compound 2 enhances affinity of the low affinity CD16a-158F Fc receptor to human IgG1 antibody. Molecular assays show that Compound 2 can significantly improve CD16a-158F Fc receptor binding to IgG1 by 4.7-fold over control compound. Experiments represent a minimum of duplicate wells. P value was generated using the Student's T-test.

FIGS. 3A-3B. Compounds 1 and 2 enhance CD16a Fc receptor activation in the Jurkat-CD16a-158F (JKT-CD16a) luciferase reporter cell line in the presence and absence of IgG1, demonstrating usefulness in antibody and non-antibody mediated immune-effector activity (FIG. 3A). Experiments represent a minimum of triplicate wells. CD16a activation is demonstrated by enhanced secretion of activation cytokine markers IFNγ and TNFα into the medium of Jurkat-CD16a cells but not CD16a-null parental Jurkat (JKT) cells FIG. 3B). P values were generated comparing compound treated vs vehicle for each group using the Student's T-test.

FIGS. 4A-4C. Compound 2 activates primary human peripheral blood mononuclear cells (PBMC), human natural killer (NK) and mouse splenocyte (SPL) effector cells and enhances antibody dependent (ADCC) (FIG. 4A) as well as antibody independent (AICC) cellular cytotoxicity (FIG. 4B) against tumor target cells. Treatment of primary NK cells with Compound 2 resulted in production of downstream CD16a Fc receptor activation markers TNF, IFNγ, GM-CSF and granzyme B protein (FIG. 4C) supporting the evidence of immune effector-mediated cell activation and subsequent target cell killing. Similar cytokine production was found in human PBMC and mouse SPL stimulated cultures. Experiments represent a minimum of triplicate wells. P values were generated using the Student's T-test.

FIGS. 5A-5B. Compound 2 was tested in vivo and shown to be well tolerated at over ten times the efficacious dose calculated from in vitro studies (FIG. 5A). Compound 2 significantly activates serum TNF-α steady state protein levels in vivo (FIG. 5B). Experiments represent a minimum of triplicate wells. P values were generated using the Student's T-test.

FIG. 6. Compound 2 activates immune-effector cells within PBMC cultures that in turn can kill SARS-COV-2 virally infected sib PBMC cells and reduce viral load. Experiments represent a minimum of triplicate wells.

FIGS. 7A-7 B. Structure activity relationship (SAR) screening reveals key active regions on Compounds 1, 2, 3 and 4 scaffolds (FIG. 7A). Particular compounds tested are shown in FIG. 7B. These data demonstrate the use of Compound 1, 2, 3 and 4 scaffolds or analogs thereof for developing additional compounds that can enhance CD16a Fc receptor-IgG antibody binding and activate or block the CD16a pathway in immune-effector cells for therapeutic applications. Experiments represent a minimum of triplicate wells. P values were generated using the Student's T-test.

FIG. 8. Additional examples of modifications at R5 and R6 that can be implemented to improve CD16a Fc receptor-IgG antibody binding and activation or suppression are shown. These can also be used as biotinylated probes (for example, Compound 34) to analyze compound-CD16a interactions in vitro and in vivo. These modifications may improve solubility, molecular activity and therapeutic activity. Methods for synthesis and final compound structure are provided below.

FIG. 9. Effect of R5/R6 modified Compound 2 on CD16a Fc receptor binding to pertuzumab. These data demonstrate the use of R5 or R6 modifications as found in Compounds 26, 27 and 33 can be incorporated into the Compound 1, 2, 3 or 4 to further enhance CD16a Fc receptor-IgG antibody binding and activate or block the CD16a pathway in immune-effector cells for therapeutic applications. Alternatively, R5 or R6 modifications as found in Compounds 29, 31 and 32 can be incorporated to suppress IgG binding to CD16a Fc receptor and potentially useful to suppress inflammation for therapeutic applications. Experiments represent a minimum of triplicate wells. P values were generated using the Student's T-test.

FIG. 10. Biologic and pharmacologic analysis of Compounds 2, 26, 27 and 33 that showed enhanced CD16a-pertuzumab binding in FIG. 9 were conducted to identify compounds with most optimal properties. Compounds and ethanol (EtOH) vehicle were tested for ADCC killing of human SKBR3 (HER2-expressing breast cancer cells via the anti-HER2 pertuzumab (PTZ) antibody using human PBMCs as effector cells. As shown, compound 27 and 33 significantly enhanced ADCC killing over parental Compound 2 (P<0.0000075). Wells with cells and PTZ without PBMCs or compound (first bar) were used as a negative control. Experiments represent a minimum of triplicate wells. P values were generated using the Student's T-test.

FIG. 11. Schematic synthesis of Compound 27.

FIG. 12. In vivo activation of NK cells via oral administration of Compound 27. Mice (N=3 per group) were administered Compound 27 or vehicle. 24 hours after last treatment, serum was collected and analyzed by ELISA for NK cell activation biomarkers IFNγ and TNFα. As shown, both biomarkers were significantly elevated in Compound 27 treated mice (P<0.0056). Experiments represent a minimum of triplicate wells. P values were generated using the Student's T-test.

FIG. 13. Development of CD16a-IgG binding antagonists for the utility of treating inflammatory disease. ELISAs were employed to test for compound analogs that may potentially block CD16a FcR-IgG binding. As shown, Compound 29 that was derived from Compound 2 scaffold was able to significantly suppress the binding of CD16a FcR probe to pertuzumab (PTZ) antibody that was coated in 96-well microplates. Experiments represent a minimum of triplicate wells. P values were generated using the Student's T-test.

### DETAILED DESCRIPTION OF THE INVENTION

The inventors have identified small molecular weight agents that can specifically bind to cross-species CD16a Fc-γ-activating receptors, leading to activation of NK cells and other CD16a-bearing immune-effector cells in vitro and in vivo. The small molecular weight agents enhance binding of IgG antibodies to the low (158F) and high (158V) affinity CD16a Fc-γ-activating receptor (FcR). The small molecular weight agents exert their effects by altering the dynamic structure of the FcR, leading to increased FcR-IgG binding, enhanced CD16a receptor signaling and subsequent enhanced immune-effector cell activation and target cell killing. Here, we describe the use of small molecular weight agents and their active cores that stimulate the activity of FcR, that in turn leads to immune-effector cell activation and/or enhanced IgG1 affinity binding and subsequent target cell killing (See also Bruhns P et al. 113:3716-3725, 2009). The small molecular weight agents that stimulate FcR activation and enhance IgG binding can be used for treating infectious disease, cancer, and other humoral-immunosuppressed diseases. We also teach the use of compounds that may inhibit CD16a activation using the compositions containing the critical backbone scaffold for CD16a binding without stimulatory domains that can suppress CD16a activation in immune-effector cells involved in inflammatory diseases, such as but not limited to Compound 37.

The identified small molecular weight agents are capable of binding and activating rodent and human CD16a Fc receptor expressing immune-effector cells via direct binding to the CD16a Fc-γ-activating receptor (FcR). Moreover, these compounds are also capable of enhancing IgG-type antibody binding to high and low affinity CD16a receptors, thereby increasing their antibody-mediated killing mechanisms against antigen expressing target cells (Rugo H S, et al. J Clin Oncol 37:1000, 2019). Alternatively, non-stimulatory compounds offer the opportunity to block CD16a activation thereby suppressing immune-effector cell activation and unwanted inflammatory responses. While not wanting to be limited to any particular theory or mechanism of action, applicants believe that the active small molecular weight compounds engage with CD16a FcR on immune-effector cells, including macrophages, NK cells and any other cell subtype expressing CD16a FcR. They alter the dynamic structure of CD16a, leading to altered receptor signaling and immune-effector cell activation as well as enhanced affinity to IgG Fc domains, either or both leading to enhanced humoral immune cell killing responses against target cells. In addition, one or more of these compounds can be administered to patients to enhance their humoral immune response against infectious disease pathogens and dysregulated cells, including cancer. Alternatively, these compounds can be modified to bind and block CD16a activation leading to suppressed immune-effector cell activation and their associated downstream inflammatory cascade. This application is useful for suppressing unwanted inflammation associated with inflammatory diseases commonly known by those skilled in the art.

Additional analogs of the above-described compounds may be similarly identified that further improve their in vivo therapeutic activity by enhanced CD16a FcR binding and activation or blockade and deactivation, as well as their pharmacokinetic (PK) and pharmacodynamic (PD) properties. Such enhancements can improve therapeutic efficacy and patient tolerability, all of which can be further determined through preclinical and human testing.

Some of the methods, compositions, and kits may be divided into three categories. In one category, compounds are developed for use as therapeutics. It is well known in the art that stronger antibody binding affinity to CD16a Fc receptor, especially on the low affinity human CD16a-158F FcR allotype, leads to improved therapeutic response against target cells expressing an antibody's specific antigen (Stavenhagen, J B et al. Cancer Res 67:8882-8890, 2007). Moreover, it is also known in the art that activated NK cells are effective at killing virally infected and dysregulated cell types (Minetto, P, et al. Front Immunol 10:1-10, 2019; Market M, et al. Front Immunol 11:1-23, 2020). The compounds described herein are able to both activate NK cells and enhance antibody binding to CD16a Fc receptors. In yet another category, it is known by those in the art that dysregulated inflammatory cells expressing CD16a can lead to unwanted inflammation causing a variety of inflammatory diseases (Cooper D L, et al. PLOS 7: e28918, 2012). The use of analogs employing the compositions and methods described here enable the development of compounds that can bind and block immune-effector cell activation and its downstream inflammatory cascade. Finally, another category is the use of active compounds as molecular biology reagents to enhance CD16a-IgG antibody binding in ELISA and other binding formats to facilitate the screening of inhibitors or activators of CD16a FcR-IgG antibody binding and subsequent pathway suppression/activation. These compounds can be employed in kits or as a stand-alone assay-enhancing reagent.

For cancer and infectious disease therapeutic use, compounds can be administered alone for AICC therapy or in combination with experimental or regulatory approved anti-cancer/anti-viral antibodies that utilize ADCC or ADCP mechanism of action. Certain anti-cancer and anti-viral antibodies that may benefit from the enhanced CD16a activation and/or binding of active compounds include but are not limited to anti-cancer agents: rituximab, trastuzumab, trastuzumab emtansine, cetuximab, YP218, ocrelizumab, daratumumab, elotuzumab, alemtuzumab, necitumumab, pertuzumab, obinutuzumab, nivolumab, ipilimumab, pembrolizumab, ofatumumab, panitumumab, tremelimumab, mosunetuzumab, penpulimab, amivantamab, margetuximab, naxitamab, tafasitamab, inebilizumab, isatuximab, and durvalumab. Additionally, compounds can be administered with anti-infectious disease antibodies including, but not limited to: nirsevimab, tixagevimab, sotrovimab, regdanvimab and the combination antibody therapy casirivimab plus imdevimab. Those antibodies that have improved humoral immune function (ADCC or ADCP) may be used for combination therapy using the optimal amount of compound.

For therapeutic applications, compounds can be administered orally, sublingually, buccally, topically or parenterally via intramuscular, intravenous or subcutaneous methods.

Compounds can be formulated to support these various routes of administration. They may be delivered via liposomal formulation or other formulations. Conventional liposomes consist of a lipid bilayer composed of cationic, anionic, or neutral (phospho) lipids and cholesterol, which encloses an aqueous volume. Suitable compositions of liposomes include without limitation, the guanidinium-cholesterol cationic lipid bis(guanidinium)-tren-cholesterol (BGTC) combined with the colipid dioleoyl phosphatidylethanolamine (DOPE). Another example of a suitable liposome formulation is the aminoglycoside lipid dioleyl succinyl paromomycin (DOSP) associated with the imidazole-based helper lipid MM27. Liposomes can be sterically stabilized, using, for example, polyethylene glycol to coat a liposome. Liposomes can be ligand-targeted, if desired. Suitable ligands include antibodies, peptides, carbohydrates, and proteins.

Yet other formulations for parenteral and non-parenteral administration include but are not be limited to alpha-tocophenol, cetyl alcohol, cetearyl alcohol, corn oil mono-diglyceride, cremophor EL, enteric acrylic resins, ethanol, ethyl oleate, glycerol, isopropyl palmitate, lauryl lactate, liposomes, mono-/diglycerides of caprylic/capric acids, oleic acid, PEG, phosphate buffered saline, polysorbate 80, propylene glycol, sorbitan monocleate, sulfonamides, and trehalose.

In other embodiments of the invention, we provide methods and compositions to develop compounds that can bind and activate either allotype of CD16a-FcR and/or enhance IgG antibody-CD16a affinity binding and downstream activation.

In some embodiments, the compound is tested for the ability to enhance CD16a FcR binding and activation of CD16a FcR-expressing cells. CD16a FcR activation can be monitored by downstream pathway signaling and measurement of downstream markers such as but not limited to IL-1B, IFN-γ, TNF-«, GM-CSF, and granzyme B. These can be tested in vitro or in vivo.

In yet other embodiments, analogs of Compound 1a and 1b, 2a and 2b, 3 as well as Compound 4a and 4b are tested for improved CD16a FcR-IgG antibody binding and/or CD16a FcR mediated cellular activation of pathway expressing cells, while minimizing cellular toxicity in vitro or in vivo screening for example. These are done using a variety of methods known by those skilled in the art.

In yet another embodiment, compounds have modified regions to improve the optimal binding of CD16a FcR to IgG antibody and/or to enhance the humoral immune response of test antibodies using any method used by those skilled in the art to measure ADCC and/or ADCP.

In yet another embodiment, the compound contains the backbone of Compound 1a and 1b, 2a and 2b, or 3 as well as Compound 4a and 4b while retaining chemical groups at R1, R2, R3 and R⁴ with modified chemical groups at R5 and R⁶ that can be empirically tested for enhancing CD16a FcR-IgG antibody binding and/or immune-effector cell activation or suppression.

In one method for measuring the ability of a compound to be effective in enhancing FcR-antibody binding or CD16a-expressing cell activation; the compound is tested for direct binding of IgG antibody to CD16a FcR via ELISA, immunohistochemistry or other methods known to those skilled in the art. Compounds with 25% or greater binding are suitable for further testing.

In some embodiments, functional methods are used to measure the activity of Compound 1a and 1b, 2a and 2b, or 3 as well as Compound 4a and 4b or derived analogs in the presence or absence of antibody using ADCC, ADCP, AICC or the production of downstream cytokines such as IL-1B, IFNγ or TNFα, where the cells can be cell lines or primary NK, myeloid, PBMC or SPL cells. The term “activity” generally refers to a 10% or greater change in ADCC, ADCP or AICC target cell killing or cytokine production when compound is incubated with cells with or without antibody as compared to cells without compound. It may, depending on the cell type, antibody and compound used, also refer to a change of at least 5%, 15%, 20%, 25%, 30%, 35%, 40%, 45%, 50%, 55%, 60%, 70%, or 75%.

Compounds can be screened for improved pharmacokinetic (PK), pharmacodynamic (PD) or pharmacologic (PL) activity. In some embodiments, the compound is added to cells in vitro and effector cell activation is tested for target cell killing in the presence or absence of antibody. Compounds that have enhanced killing with or without antibody are now suitable for therapeutic testing using in vivo models. In another embodiment, compounds can be incubated in microsomes from various species to determine compound modifications that may affect such compound(s) to have a higher metabolic stability. The effect may, depending on the compound used, be a change of at least 5%, 10%, 15%, 20%, 25%, 30%, 35%, 40%, 45%, 50%, 55%, 60%, 70%, 75% or greater.

Other embodiments employ compound 1a, 1b, 2a, 2b, 3, 4a and/or 4b to bind and block CD16a activation and signaling that in turn leads to decreased downstream inflammatory signaling via persistent cytokine production that is associated with inflammatory diseases.

Various terms and terminology (“terms”) relating to aspects of the enclosed description are used throughout the specification and claims of this document. Such terms are to be given their ordinary meaning in the art unless otherwise specifically indicated. Other specifically defined terms are to be construed in a manner consistent with the definitions provided.

As used in this specification and the appended claims, the singular forms of “a,” “an,” and “the” also include plural references unless the content clearly specifically dictates otherwise. As example, reference to “a cell” may include a combination of two or more cells, and the like. Reference to “a probe” may include a poly-histidine tagged human or rodent CD16a-FcR, CD32a FcR or an independent probe to monitor FcR-antibody binding, whereby 2-10 histidine codons are engineered at the mature N- or extracelluar C-terminal domain of the FcR. These probes are referred to as CD16a-HIS or CD34a-HIS. Reference to “the cellular activation” may include effect of compound on CD16a FcR expressing cells via any analytical method known by those skilled in the art.

The term “about” as used when referring to quantified values such as an amount, a period of time, and/or the like, is meant to encompass variations of up to ±9% from the specified value, as such variations are appropriate to carry out the disclosed methods. Unless otherwise indicated, all values expressing quantities of reagents, such as molecular weight, molarity, reaction conditions, percentage and so forth used in the specification and claims are to be understood as being quantified in all instances by the term “about.” Accordingly, unless indicated to the contrary, the numerical values as set forth in the following specifications and listed claims are approximations that may vary depending upon the desired properties of the composition agent and/or methods sought to be obtained by the present invention. At the very least, and not as an attempt to limit the scope of the application, each numerical value should at least be valued by the reported significant digits and through ordinary rounding methods known by those skilled in the art.

The term “antibody” as used is meant in a broad sense and includes immunoglobulin (also referenced as “Ig”) or antibody molecules including polyclonal antibodies (also referenced as pAbs), monoclonal antibodies (also referenced as mAbs) including murine, rat, primate, human, humanized and chimerized mAbs, and full-length bispecific or trispecific antibodies. In general, antibodies are proteins or polypeptide chains that bind to a specific antigen. An antigen is a structure that is specifically recognized by a given antibody. Canonical antibodies comprise heterotetramer glycosylated proteins, composed of two light chains and two heavy chains lined through a complex of disulfide and hydrogen bonds. The term “its disulfide bridge” refers to the disulfide bridge contained within the heavy chain hinge region, which is commonly known by those skilled in the art. Each heavy chain has a variable domain (variable region) (VH) followed by a number of constant domains (referred to as the Fc domain). Each light chain has a variable domain (VL) and a constant domain; the constant domain of the light chain is aligned with the first constant domain of the heavy chain and the light chain VL is aligned with the variable domain of the heavy chain. Antibody light chains of any species are assigned to one of two distinct types based on their amino acid sequences within their constant domains, namely kappa (κ) and lambda (λ).

Immunoglobulins are categorized as classes or isotypes, depending upon the type of Fc domain namely IgA, IgD, IgE, IgG and IgM, which depend on the sequences contained within their heavy chain constant (Fc) domain. The IgG isotypes are further comprised of subclasses as the isotypes IgG₁, IgG₂, IgG₃ and IgG₄.

“Specific binding” or “specifically binds” refers to the binding of an antibody to an antigen (including sequences contained within an antibody itself) with greater affinity than for other antigens. Typically, a specific antibody or antigen-binding fragment binds the target antigen with an equilibrium dissociation constant Kp of about 5×10⁻⁸ M or less. In other instances, it refers to the binding of an antibody Fc domain to a CD16a, CD32a or CD64 Fc receptor. The equilibrium dissociation constants Kp can range depending on the Fc receptor and its allotype.

The term “allotype” refers to the amino acid sequence of CD16a or CD32a Fc receptors that have been shown to differ in the human population with each having a different binding affinity to IgG Fc. For CD16a it typically involves the amino acid at position 158 that contains a valine (V) or phenylalanine (F), as commonly known by those skilled in the art.

The term “affinity” refers to the measurement of antibody on versus off rate of antigen binding to the variable domain as well as the measurement of antibody on vs off rate binding of the antibody Fc domain to Fc receptors, including CD16a, CD32a and CD64.

An “antibody derivative” means an antibody, as defined above, that is modified by covalent attachment of another molecule such via peptide chemistry (i.e., amidation, etc.), genetic fusion and/or via post translational moieties (i.e., glycosyl, acetyl and/or phosphoryl) not typically associated with the antibody, and the like. This may include the chemical fusion of a compound taught here with an antibody to enhance its FcR binding activity.

The term “CD16a dynamic structure” refers to any change in structure that can affect CD16a-expressing immune-effector cell humoral function, activation, suppression and/or CD16a-IgG antibody binding.

“Fc domain” refers to any antibody sequence C-terminal to and including the antibody hinge disulfide region. This region includes the binding domain of the CD16a, CD32a and CD64 Fc receptors and is known by those skilled in the art.

An “antigen” is an entity to which an antibody or antibody fragment specifically binds. This includes binding to an antibody or protein of interest.

The term “mature” refers to an extracellular membrane or secreted protein wherein N-terminal secretion signal sequences are cleaved, a concept well known by those skilled in the art.

The terms “cancer,” “malignant,” and “tumor” are well known in the art and refer to the presence of cells with unregulated cell growth and morphological features different than a normal cell type of similar origin, also referred to as “dysregulated cells”. Malignant refers to those cancer cells capable of causing morbidity and/or mortality. As used, “cancer and tumor” includes premalignant and malignant types.

The terms “viral,” “pathogenic,” and “infected” are well known in the art and refer to the presence of cells or subjects that have been infected with disease causing microbes or viral particles.

The term “inflammation” or “inflammatory disease” refers to a patient's disease whereby inflammatory cells are dysregulated causing inflammation to one or more organs of the body.

As used, the term “soluble” refers to a protein or non-protein agent that is not attached to the cellular membrane of a cell. For example, an agent that is soluble may be shed, secreted or exported from normal or cancerous cells into biological fluids including serum, whole blood, plasma, urine or microfluids of a cell, including tumors. A natural membrane bound Fc receptor such as CD16a, CD32a or CD64 or one containing a poly-histidine tag can be engineered to be soluble and functional by incorporating a stop codon prior to the receptor's transmembrane domain.

The term “detector agent” or “detectable agent” refers to any agent that can be linked to an antibody or FcR and detected via devices commonly used in the field. These include but are not limited to fluorophores, enzymes and enzyme substrates, radionuclides, heavy metals and colorimetric dyes and substrates that can be detected via methods such as but not limited to densitometry, spectrophotometry, luminescence, microscopy, radiography and scintillation. Additional reagents may be needed to develop the detectable signal.

The term “biomarker” refers to immune-effector cell produced proteins that are enhanced or suppressed by treatment with a compound. In particular interferon-gamma (also referred to as IFN-gamma or IFNγ), tumor necrosis factor-alpha (also referred to as TNF-alpha or TNFα) and granzyme B are known biomarkers to be produced by CD16a Fc receptor stimulated immune-effector cells, including NK cells.

The “level” of a specified protein or compound including CD16a FcR induced proteins from target cells such as but not limited to granzyme B, IFNγ or TNFα, as used, refers to the level or levels of the protein or compound as determined using any method known in the art for the measurement of protein and/or compound levels in vitro or in vivo in response to treatment with a compound. Such methods include gel electrophoresis, capillary electrophoresis, high performance liquid chromatography (HPLC), thin layer chromatography (TLC), hyperdiffusion chromatography, fluid or gel precipitation reactions, absorption spectroscopy, colorimetric assays, spectrophotometric assays, flow cytometry, immunodiffusion (single or double), solution phase assay, immunoelectrophoresis, Western blotting, radioimmunoassay (RIA), enzyme-linked immunosorbent assays (ELISAs), immunofluorescent assays, fluorescence resonance energy transfer (FRET), Förster resonance energy transfer, electrochemiluminescence immunoassay, and the like. In one embodiment, the level of IFNγ and/or TNF& is determined using probe-based techniques, as described in more detail. In another embodiment, the level of a compound is determined using chromatographic and ELISA-based techniques.

The term “humoral immuno-suppression, immunosuppression, or humoral immune suppression” refers to any antibody, antibody fragment, bispecific or trispecific antibody or immune-effector cell (i.e., NK, myeloid, monocyte, dendritic cells, etc.) whose maximal biological response is muted. FcR receptor allotype as well as lack of immune-effector cell activation have been reported to affect the biological activities of these humoral immune components including ADCC, ADCP, AICC and/or PK, PD and PL profiles.

The term “antibody drug conjugate (ADC)” refers to any antibody that is conjugated or fused to a compound that results in the antibody's enhanced activity, including enhanced binding to FcR that results in enhanced ADCC and ADCP.

The term “bispecific or trispecific antibody (BSP)” refers to any antibody that can bind two or more different antigens. A BSP can comprise at least but not limited to two full length antibodies, a full-length antibody or a single chain antibody wherein each one binds to different antigens or different epitopes on the same antigen.

The term “antibody dependent cellular cytotoxicity (ADCC)” refers to an in vitro or in vivo process where an antibody can bind to an antigen on a surface of a cell then engage with immune-effector cells via sequences within the antibody's Fc domain that in turn results in the immune-effector cells releasing toxins that can kill bound cell.

The term “antibody independent cellular cytotoxicity (AICC)” refers to an in vitro or in vivo process where a CD16a-expressing cell is stimulated via non-antibody CD16a FcR activation that in turn results in the CD16a-expressing cells binding dysregulated or infected cells via natural cell surface receptors, resulting in the release of toxins that can kill bound cell.

The term “antibody dependent cellular phagocytosis (ADCP) and opsonization” refers to a process where an antibody can bind to an antigen on a surface of a dysregulated cell then engage with immune cells via sequences within its Fc domain that in turn results in immune cells engulfing, consuming and ultimately killing antibody bound cell.

The term “screening” may refer to testing compounds and measuring enhanced or diminished biological response by monitoring ADCC, AICC, ADCP or other immune-effector cell activation, with the intent of identifying compounds with such activities. Other references refer to looking for compounds that can block or enhance CD16a Fc receptor binding to IgG antibody. The term may be used in other contexts in which a large number of test elements is being assayed to determine which among the test elements has a certain property. Similarly, it can be used to refer to the assaying of patient samples for those having a particular property.

The term “significant(ly)” refers to statistical results where the P value as determined by any number of programs including the Student's T-Test when its value is less than 0.05.

The term “pharmacokinetic (PK)” refers to the time that a compound maintains its steady-state concentration when administered to a subject.

The term “pharmacodynamic (PD)” refers to the study of the biochemical and physiological effects of a compound and its mechanisms of action(s), including the correlation of their actions and effects with their structure when administered to a subject.

The term “pharmacologic (PL)” refers to the known effect a compound has on managing or killing a disease cell in vitro or in vivo.

The term “maximum tolerated dose (MTD)” refers to the maximum dose that a compound can be administered to a patient or test animal before they experience toxic signals. In test animals, MTD is typically defined as loss of greater than 10% body weight.

The term “sample” refers to a collection of similar fluids, cells or tissues isolated from a subject, as well as fluids, cells or tissues present within a subject. Fluids may include biological fluids that include liquid solutions contacted with a subject or biological source, including cell and organoid culture medium, urine, salivary, lavage fluids and the like.

The term “control sample,” as used, refers to any clinically or non-clinically relevant control sample, including, for example, a sample from a healthy subject not afflicted with a particular cancer type or a cell that is different from its parental cell.

The term “control level” refers to an accepted or pre-determined level of a protein or non-protein agent that is used to compare with the level of the same agent in a sample derived from a subject or used in in vitro assays.

As used, “a difference” between signal of two compounds that may be of common backbone is generally any difference that can be statistically determined using statistical methods commonly used by those skilled in the art and at a minimum a difference of 10% or greater as compared to control. It may, depending on the compound and the assay used also refer to a change of at least 5%, 15%, 20%, 25%, 30%, 35%, 40%, 45%, 50%, 55%, 60%, 70%, or 75%.

The term “inhibit” or “inhibition of” means to reduce by a statistically measurable amount, or to prevent entirely.

The term “functional,” in the context of a compound in the presence or absence of an antibody used in accordance with the methods described, indicates that the compound is capable of enhancing or blocking binding of antibody and/or activation of immune-effector cells, respectively, leading to enhanced killing of target cells in vitro or in vivo and/or production of downstream proteins or suppression of immune-effector cell inflammatory pathway signaling

The term “pharmaceutically acceptable” refers to a substance that is acceptable to administer to a patient from a pharmacological as well as toxicological aspect and is manufactured using approaches known by those skilled in the art. These include agents approved by a regulatory agency of the Federal or a state government or listed in the U.S. Pharmacopeia or other generally recognized pharmacopeia for use in animals and humans. The term “pharmaceutically compatible ingredient” refers to a pharmaceutically acceptable diluent, adjuvant, excipient or matrix vehicle with which an anti-cancer agent is administered. “Pharmaceutically acceptable carrier” refers to a matrix that does not interfere with the effectiveness of the biological activity of the active ingredient(s) and is nontoxic to the host.

The term “formulation” refers to solvents, excipients or matrix vehicles that a compound is added to in order to support its administration to patients.

The terms “patient” and “subject” are used interchangeably to refer to humans and other non-human animals, including veterinary subjects, that receive a therapeutic agent treatment. The term “non-human animal” includes all vertebrates. In one embodiment, the subject is a human.

The terms “effective amount” and “therapeutically effective” are used interchangeably and, in the context of administering a pharmaceutical agent at an amount that is sufficient to produce an enhanced clinical outcome in a patient. An effective amount of an agent is administered according to the methods described here in an “effective regimen.” The term “effective regimen” refers to a combination of amount of the agent and dosage frequency adequate to accomplish an enhanced clinical outcome for a patient with a particular cancer. Enhanced efficacy is an improved clinical outcome when a patient is administered an agent that is capable of overcoming morbidity better than a parental compound or an agent that can enhance the clinical outcome of an effective regimen. As in context here, effective amount refers to the amount of compound required to demonstrate efficacy or a difference when compared to no compound.

“Therapeutic agents” are typically substantially free from undesired contaminants. This means that an agent is typically at least about 50% w/w (weight/weight) pure as well as substantially free from interfering proteins and contaminants.

The term “target cell” refers to a eukaryotic or prokaryotic cell or population of cells that expresses antigen for a specific antibody or antibody containing moiety and is bound by the CD16a Fc receptor expressing cells through direct antibody engagement for ADCC or ADCP activity or indirect antibody interaction by AICC.

The term “immune-effector cell” refers to any cell line or natural cell including but not limited to NK, myeloid, monocytes, neutrophils, dendritic cells that may confer antibody dependent cellular cytotoxicity (ADCC) or phagocytosis (ADCP or opsonization) upon binding to antibody-bound target cell as well as confer antibody independent cellular cytotoxicity (AICC) upon CD16a FcR stimulation. Natural cells may be purified or present in mixture in the form of peripheral blood mononuclear cells (PBMCs). Cell lines may naturally express CD16a or be engineered to express CD16a.

The term “dysregulated cell” refers to any cell that is deemed abnormal to parental cells. These include transformed cells, malignant cells, virally infected cells, autonomously growing cells via autoregulation, or prokaryotic pathogens.

The term “humoral response” refers to ADCC or ADCP of target cells by immune-effector cells in the presence of test antibody. The term “cellular response” refers to AICC of target cells by immune-effector cells in absence of test antibody.

The term “compound” refers to any compound or formula conceived compound described within this document that can bind and alter the activity of CD16a FcR. In particular it refers to the use of the cholestane-based lanosta-8,24-dien-3β-ol (Compound 1a); 9β-19-cyclo-24-lanosten-3β-ol (Compound 2a) and bisnoralcohol-derived compounds (Compounds 3) as well as analogs consisting of their three cyclohexyl (rings A, B, C) and cyclopentyl (ring D) ring backbone structure as scaffolds (Compound 4a). The term “compound” also refers to any compound or formula conceived compound described within this document that can bind and enhance or block the activity of CD16a FcR, including Compound 1a, 1b, 2a, 2b as well as analogs consisting of their three cyclohexyl (rings A, B, C) and cyclopentyl (ring D) ring backbone structure as scaffolds (Compounds 3, 4a or 4b).

The term “analog” refers to any compound derived from Compounds 1a, 2a, 3, 4a, 1b, 2b, or 4b, where single or multiple modifications are made at R1, R2, R3, R₄, R5 and/or R6.

The term “food” refers to a material consisting essentially of protein, carbohydrate and/or fat, which is used in the body of an organism to sustain growth, repair and vital processes and to furnish energy. Foods may also contain supplementary substances such as minerals, vitamins and condiments. See Merriam-Webster's Collegiate Dictionary, 10th Edition, 1993. The term food includes a beverage adapted for human or animal consumption. As used herein a “food additive” is as defined by the FDA in 21 C.F.R. 170.3 (e) (1) and includes direct and indirect additives.

The term “dietary supplement” refers to a product (other than tobacco) that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients.

### Therapeutic Compounds, Kits, and Methods for Enhancing Humoral Immune Mediated Therapy

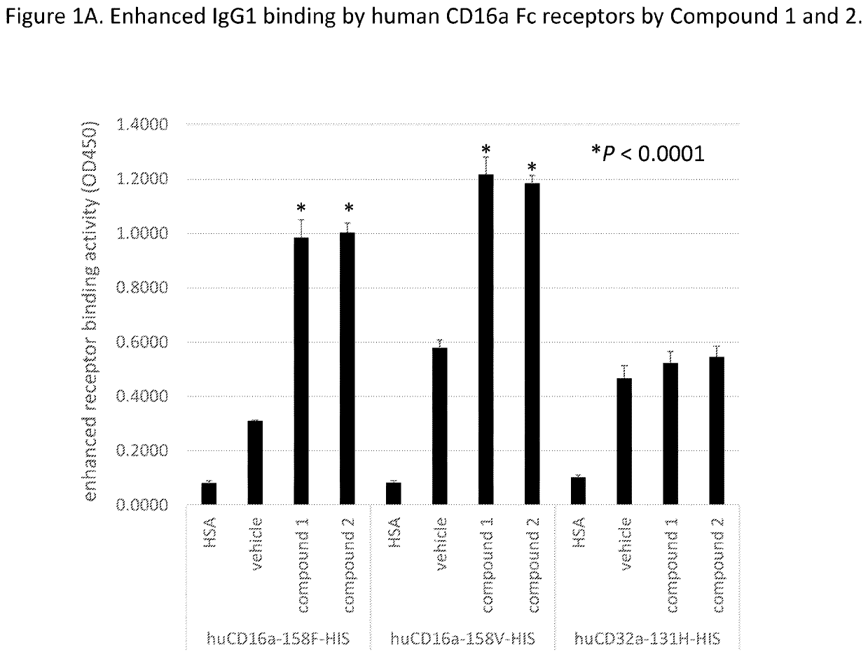

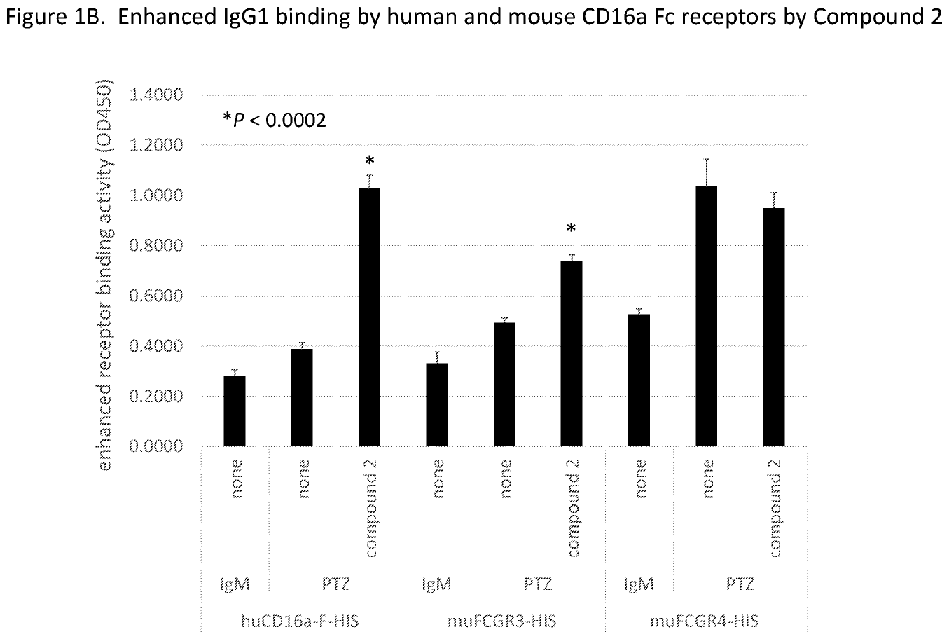

Provided here are compositions, kits and methods for identifying small molecular weight compounds that can effectively enhance or suppress CD16a Fc receptor binding to IgG antibody and/or activate immune-effector cell response against pathogens, virally-infected or dysregulated cancer cells or suppress dysregulated inflammatory cells. Examples are schematically shown in FIGS. 1-11. Kits are composed of a compound whose chemical composition is taught via the formulas within this invention and the schematic methods for synthesizing the propriety compositions as described in Example 3. Active compounds can be identified using molecular CD16a FcR-IgG antibody binding and/or effector-cell activation or suppression assays as described throughout the Examples below.

In the methods for identifying activating compounds via cell-based screening, a compound is added to a culture of target cells that express target antigen of a test antibody, with or without antibody. Immune-effector cells are added to cultures and humoral immune response is compared in those with compound vs those without and are monitored for humoral response using standard ADCC, ADCP or AICC killing assays. A change in at least 10% is typically considered as being a meaningful effect on ADCC, ADCP or AICC functions. Depending on the assay employed, a meaningful effect also may be defined as a change of at least 5%, 15%, 20%, 25%, 30%, 35%, 40%, 45%, 50%, 55%, 60%, 70%, or 75%.

In other methods for identifying activating compounds, a test antibody is added to a culture of target cells in which target cells naturally express antigen of test antibody. Engineered human Jurkat cells expressing the human CD16a Fc receptor (JKT-CD16a) linked to a luciferase reporter is added, and luciferase levels are monitored after 12-24 hours. A change in at least 10% is typically considered as being a meaningful effect on CD16a activation. Depending on the antibody and the assay employed, a meaningful effect also may be defined as a change of at least 5%, 15%, 20%, 25%, 30%, 35%, 40%, 45%, 50%, 55%, 60%, 70%, or 75%.

Also provided here are methods of treating a cancer subject with an active compound. For example, a patient may have a CD20, HER2, EGFR, or a mesothelin-expressing cancer such as lymphoma, breast, mesothelioma, colorectal, lung, ovarian, pancreatic, cholangio, or endometrial carcinoma. Several antibodies targeting these antigens have been reported to be more effective in patients whose immune-effector cells express the high-affinity CD16a-158V FcR allotype (Rugo H S, et al. J Clin Oncol 37:1000, 2019). Unfortunately, greater than 50% of world's population express the low CD16a-158F FcR, therefore making a compound that can enhance the binding affinity of a test antibody to CD16a-158F FcR a pharmaceutically desirable entity (Wang W, et al. Cytogenet Genome Res 152:169-179, 2017). One such antibody is the anti-CD20 rituximab, which has been found in clinical studies to have significantly less therapeutic effect in patients who are heterozygous or homozygous for the CD16a-158F allele (Treon S P, et al. J Clin Oncol 23:474-481, 2016). The use of an effective compound for this antibody is also highly pharmaceutically desirable.

In some embodiments of the methods of treating a subject with an active compound, a patient with an infectious disease or cancer may be treated with a compound alone or in combination with standard-of-care therapy. In some embodiments of the methods of treating a subject described here, a compound is administered to the subject alone. In yet another embodiment, the compound is administered in combination with standard-of-care therapy, which includes all agents considered standard-of-care for a disease indication at the time when the subject is treated.

In methods for identifying CD16a pathway blocking compounds, a test antibody is added to a culture of target cells in which target cells naturally express antigen of test antibody. Engineered human Jurkat cells expressing the human CD16a Fc receptor (JKT-CD16a) linked to a luciferase reporter is added, and luciferase levels are monitored after 12-24 hours. A suppressed signal of at least 10% is typically considered as being a meaningful effect on CD16a suppression when compared to cultures not treated with compound. Depending on the antibody and the assay employed, a meaningful effect also may be defined as a suppression of at least 5%, 15%, 20%, 25%, 30%, 35%, 40%, 45%, 50%, 55%, 60%, 70%, or 75%.

In some embodiments of the methods of treating a subject with an active compound, a patient with inflammatory disease may be treated with a compound alone or in combination with standard-of-care therapy. In some embodiments of the methods of treating a subject described here, a compound is administered to the subject alone. In yet another embodiment, the compound is administered in combination with standard-of-care therapy, which includes all agents considered standard-of-care for a disease indication at the time when the subject is treated.

In some embodiments of the methods of treatment described here, the compound consists of a chemical backbone derived from Compounds 1, 2, 3 or 4 and retains the chemical groups at R1, R2 and R3 while using formulas taught or exemplified here to modify R4, R5 and/or R6 regions.

The present methods can be combined with alternative treatments such as surgery, radiation, targeted therapy, chemotherapy, immunotherapy, use of growth factor inhibitors, or anti-angiogenesis factors. A compound can be administered concurrently to a patient undergoing an alternative treatment. A patient can also undergo alternative treatment subsequent to administration of a compound by at least an hour and up to several months, for example at least an hour, five hours, 12 hours, a day, a week, a month, or three months, prior or subsequent to administration of standard of care therapy. Some embodiments of the methods of treatment provided here involve administration of a therapeutically effective antibody that is specific for an antigen expressed by said pathogen, virus, cancer, or inflammatory mediator to the subject in addition to the compound.

In some embodiments of the methods of treatment described here, the subject may have received alternative therapy and an antibody-based standard-of-care treatment prior to administering compound.

Administration of the therapeutic agents and compounds in accordance with the methods of treatment described here may be by any means known in the art.

The amount of the therapeutic agent or compound that is effective in the treatment or prophylaxis of a cancer or non-oncologic (viral, pathogen, or inflammatory cell) mediated disease can be determined by standard clinical techniques. In addition, in vitro assays may optionally be employed to help identify optimal dosage ranges required for the compound using standard or proprietary screening methods taught here. Effective doses may be extrapolated from dose-response curves of compound derived from in vitro or animal model test systems.

For example, toxicity and therapeutic efficacy of the agents can be determined in cell cultures or experimental animals by standard pharmaceutical procedures for determining the LD₅₀ (the dose lethal to 50% of the population) and the ED₅₀ (the dose therapeutically effective in 50% of the population) values. The dose ratio between toxic and therapeutic effects is the therapeutic index and it can be expressed as the ratio LD₅₀/ED₅₀. Agents that exhibit therapeutic indices of greater than 2-fold are suitable. When an agent exhibits toxic side effects, a delivery system that targets the agent to the site of affected tissue can be used to minimize potential damage to cells in healthy tissues and, thereby, reduce side effects.

The dosing and dosage schedule may vary depending on the active drug concentration, which may depend on the needs of the subject.

### Kits to Optimize Activity of Cd16A Fcr Binding to Igg-Type Antibodies for Screening

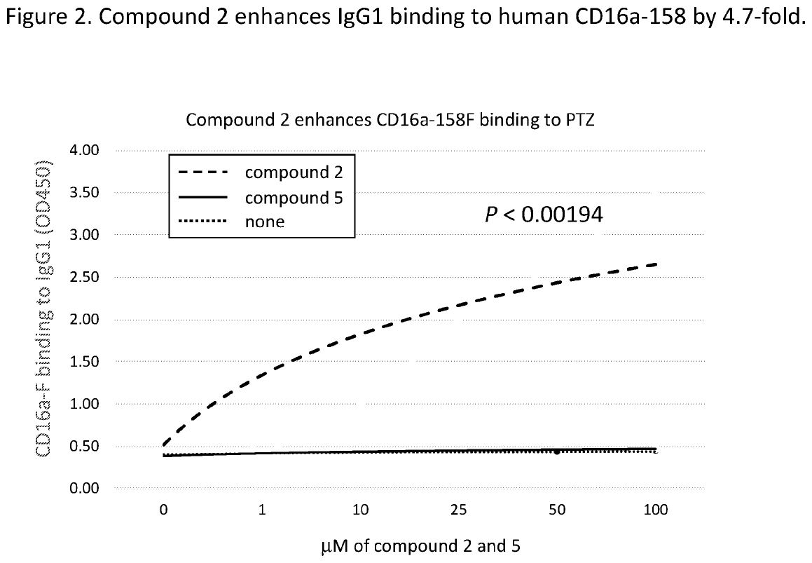

Further provided here are kits for using active compounds to enhance human or murine CD16a FcR binding to IgG antibodies for rapid screening of inhibitors or activators that can affect the pathway. It is taught here that active compositions provided in this invention are able to significantly improve CD16a FcR-IgG antibody binding in ELISA and other binding assay formats. This improvement facilitates the ability to screen for agents that can positively or negatively affect the CD16a FcR biological pathway in immune-effector cells for developing therapeutics that are useful for treating diseases whereby modulating the CD16a FcR pathway may have clinical benefits. One teaching includes the addition of an active compound (such as but not limited to Compounds 1, 2, 4, 14, 26, 27, 30, 33 or 36) to be added in high throughput screening assays using CD16a FcR and antibodies to find small or macro molecular weight inhibitors (Mandelboim O, et al. Proc Natl Acad Sci USA 96:5640-5644, 1999). Other screens may be used to screen Fc domain modified antibodies or Fc fusion proteins for enhanced CD16a FcR binding to improve pharmacologic activity (Saunders K O. Front Immunol 10:1-20, 2019). Examples of this feature are provided in FIGS. 1 and 2.

Kits may include a compound with reagents commonly used in ELISAs, or just compound alone with instructions for use in enhancing CD16a FcR-IgG antibody binding in molecular or cellular binding assays.

The above disclosure generally describes the present invention. All references disclosed here are expressly incorporated by reference. A more complete understanding can be obtained by reference to the following specific examples which are provided here for purposes of illustration only, and are not intended to limit the scope of the invention.

### Example 1—Identification of Compounds 1 and 2 for Enhancing Immunoglobulin Binding to Rodent and Human Cd16A-Fc Receptors and Immune-Effector Cell Activation In Vitro and In Vivo

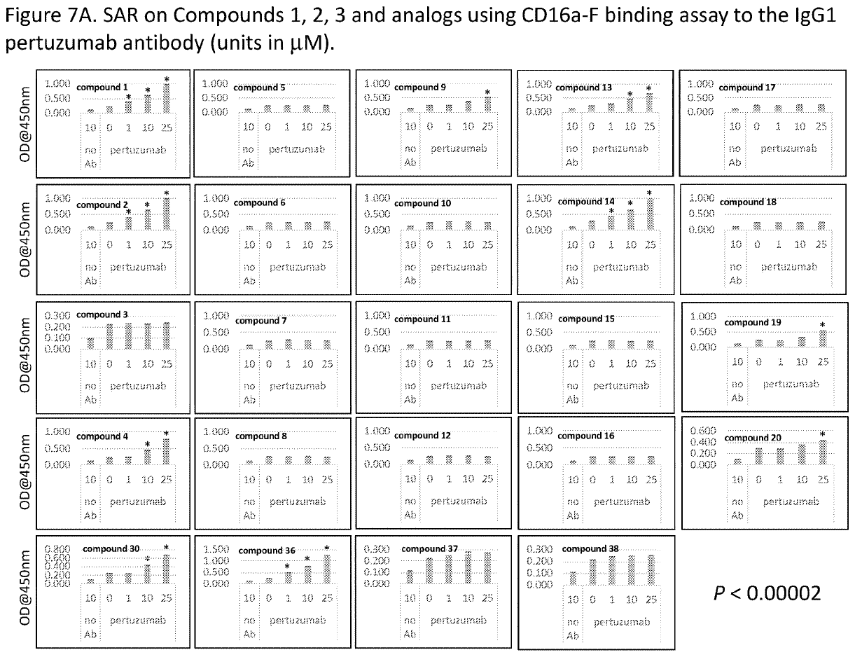

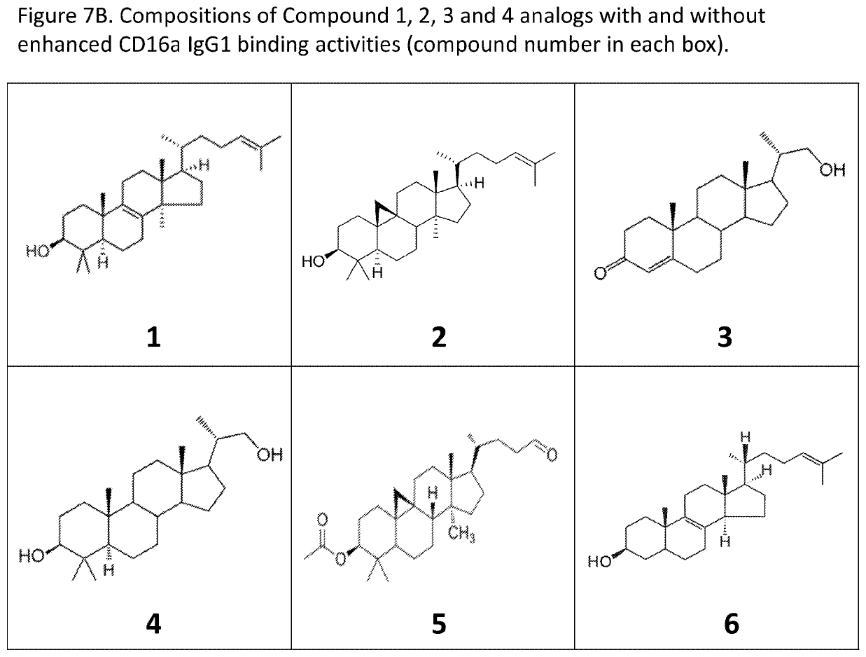

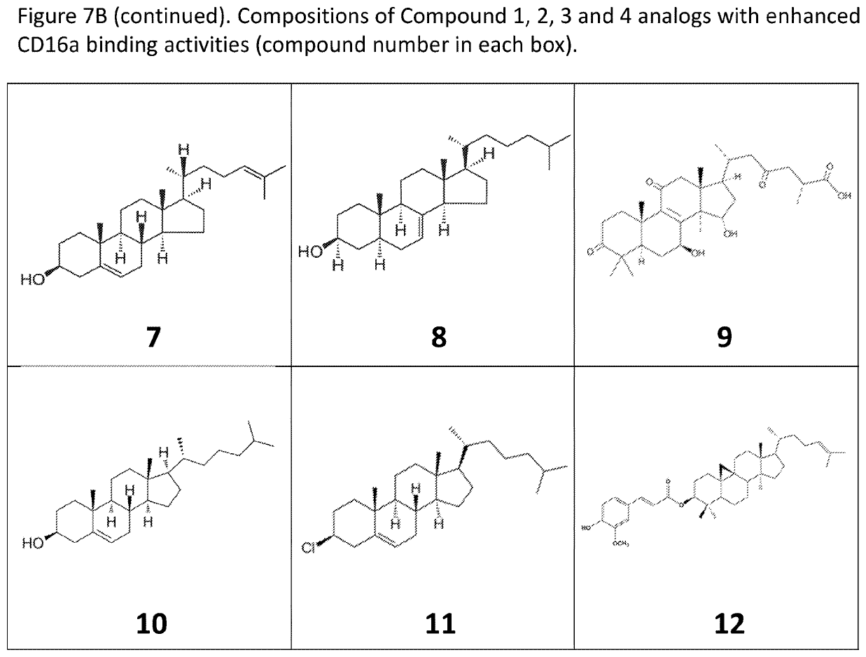

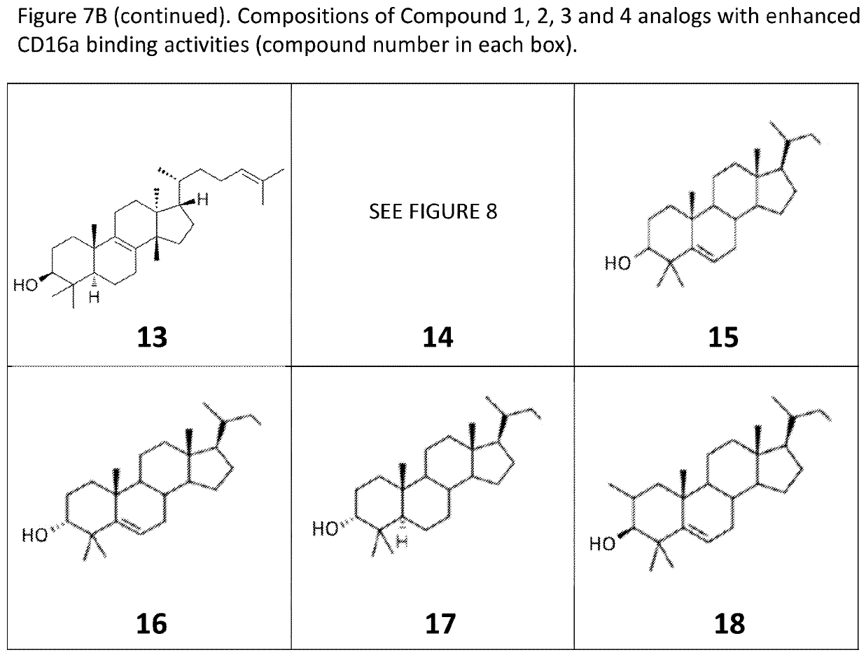

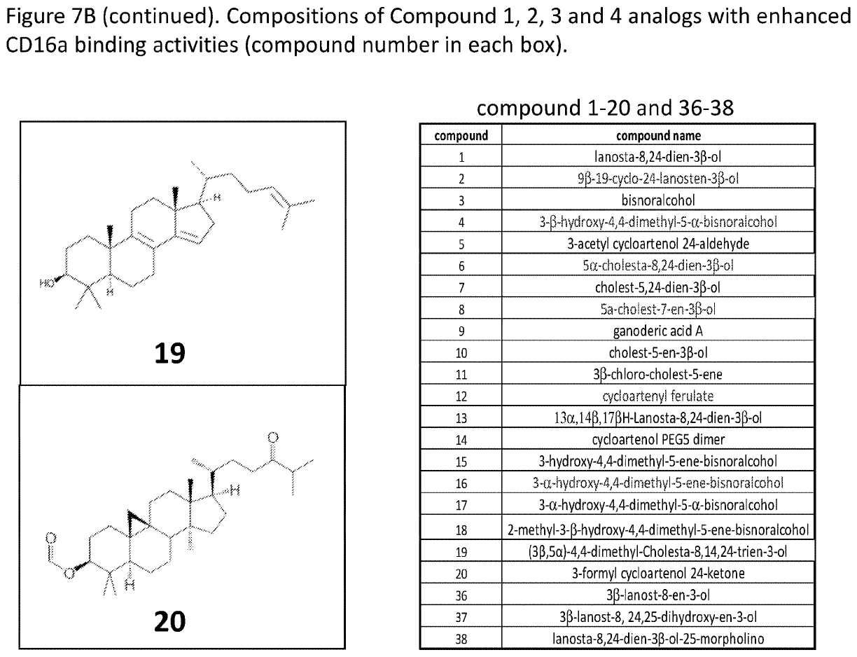

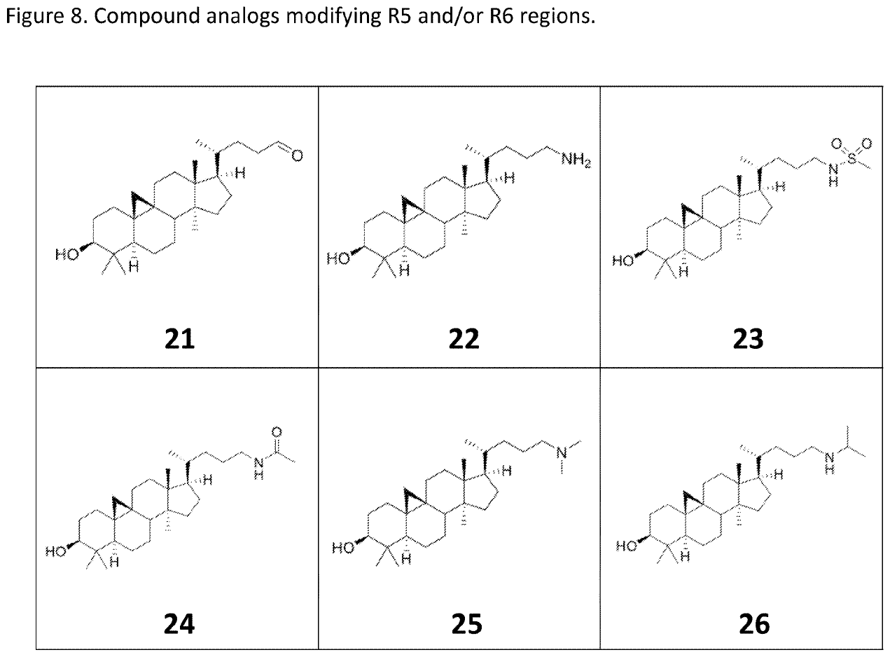

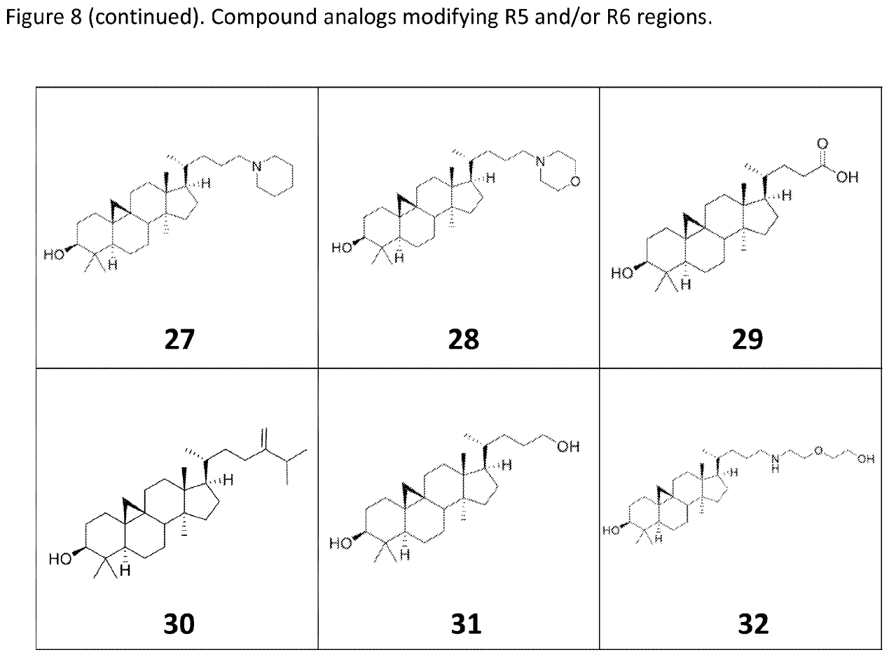

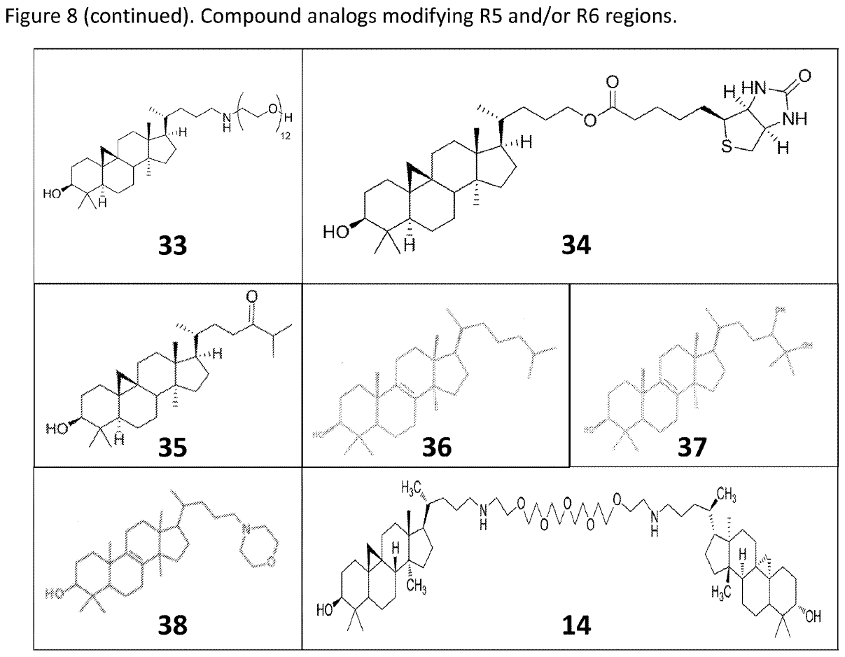

In an effort to identify compounds that could potentially bind and enhance the low affinity human CD16a-158F FcR binding to IgG-type immunoglobulins, semi-high throughput screening assays were developed to screen natural small molecule chemical libraries acquired from commercial sources. These libraries contained over 1,100 small molecular weight chemicals derived from a variety of natural sources and comprised of a vast array of chemicals with varying structures and biological properties. The primary assay employed a 96-well ELISA-based format, whereby plates were coated with 2.5 μg/mL of human IgG1 or human serum albumin (HSA), used as a negative control, and tested for CD16a-158F-HIS (SEQ ID NO: 1), CD16a-158V-HIS (SEQ ID NO: 2) or CD32a-131H-HIS (SEQ ID NO: 3) FcR binding using 2.5 μg/mL of each probe. The cDNA of each probe lacking the transmembrane domain and a C-terminal poly-histidine tag for purification was engineered into a mammalian expression vector containing the blasticidin selection marker. Vectors were stably transduced into HEK293 cells and blasticidin-resistant clones were screened for high-titer production of FcR proteins using ELISA based screening methods and an anti-poly-histidine (HIS)-horseradish peroxidase (HRP) conjugated antibody as detector probe. The secreted mature CD16a or CD32a proteins (leader sequence cleaved during secretion) were isolated from 10-day cultures grown to 1.5×10⁶ cells/mL in serum-free BalanCD HEK293 media (Irvine Scientific) and isolated over HisPur™ Ni-NTA resin columns as recommended by the vendor (Thermo Scientific). After primary probing of compound libraries, wells were washed and monitored for IgG binding via the secondary anti-HIS-HRP detector antibody (Sino Biologicals). Well signals corresponding to individual clones were quantified using a multi-well plate reader (Varioskan, ThermoFisher) at 450 nm. After screening over 1,100 compounds only one compound was found to reproducibly enhance huCD16a-158F FcR binding to IgG₁ antibody over controls. Compound analysis identified this to be the cholestane-based lanosta-8,24-dien-3β-ol (Compound 1). Structural analysis found the highly related 9β-19-cyclo-24-lanosten-3β-ol compound (Compound 2) that was subsequently acquired and tested to compare molecular and cellular activities on CD16a and CD32a FcRs with Compound 1. As shown in FIG. 1A, both Compound 1 and 2 could significantly enhance the binding of huCD16a-158F and huCD16a-158V FcRs to IgG1 antibody in contrast to huCD32a-131H FcR or HSA, demonstrating target specificity of both compounds. Compound 2 was further tested for the ability to enhance human IgG binding to other CD16a species. As shown in FIG. 1B, Compound 2 enhances binding of murine CD16a homolog 1 (FCGR3) (SEQ ID NO: 4) (muFCGR3-HIS) to human IgG1 but not the murine homolog 2 (FCGR4) (SEQ ID NO: 5) (muFCGR4-HIS). These analyses were done using similar ELISA methods as above and HIS-tagged murine FCGR3 and FCGR4 as probes (Sino Biologics) followed by anti-HIS-HRP for detection. These data identify rodent as a relevant species to test Compounds 1, 2, 3 and 4 as well as derived analogs for enhancing immune-effector activation in vivo for therapeutic confirmation and proof-of-concept. In addition, the data shown in FIG. 1 support the use of Compounds 1 or 2, as well as their related active analogs shown in FIGS. 7 and 8. Any and all compounds that are potential enhancers for CD16a FcR-IgG binding, can be employed as assay reagents in molecular biology studies to facilitate screening for compounds that can positively or negatively affect CD16a FcR-IgG antibody biological pathways for therapeutic indications involving this pathway. The application of these compounds as molecular biology assay reagents results in robust ELISA signal readouts in 3 to 5 minutes in contrast to 15 to 30 minutes required in basic CD16a FcR-IgG antibody binding formats, thus facilitating screening platforms. Experiments represent a minimum of triplicate wells. P values were generated using the Student's T-test.

To further evaluate the effect of enhanced CD16a FcR-IgG antibody binding, titration curves were generated to determine the amount of enhanced binding that Compound 2 could confer on the low affinity huCD16a-158F FcR. Briefly, ELISA plates were coated with 2.5 μg/mL of human IgG₁ and probed with 2.5 μg/mL of huCD16a-158F-HIS FcR in the presence of varying concentrations of Compound 2 or an inactive analog (Compound 5, see FIG. 7). After probing, plates were washed and secondarily probed with anti-HIS-HRP for detection. Wells were quantified using a multi-well plate reader (Varioskan) at 450 nm. As shown in FIG. 2, Compound 2 can improve CD16a-158F-IgG antibody binding 4.7-fold over control Compound 5 and suggests a significant improvement in affinity binding over CD16a-158F alone. This improvement is calculated to increase huCD16a-158F affinity for IgG₁ binding from approximately 1 μM to 200 nM making its affinity similar to that of the high affinity huCD16a-158V FcR allotype whose published affinity is approximately 415 nM (Nordstrom J L, et al. Breast Cancer Res 13:1-14, 2011), an unexpected finding and a feature that is highly desirable in developing CD16a-based therapies (Bowles J A, et al. Blood 108:2648-2654, 2006). Experiments represent a minimum of triplicate wells. P values were generated using the Student's T-test.

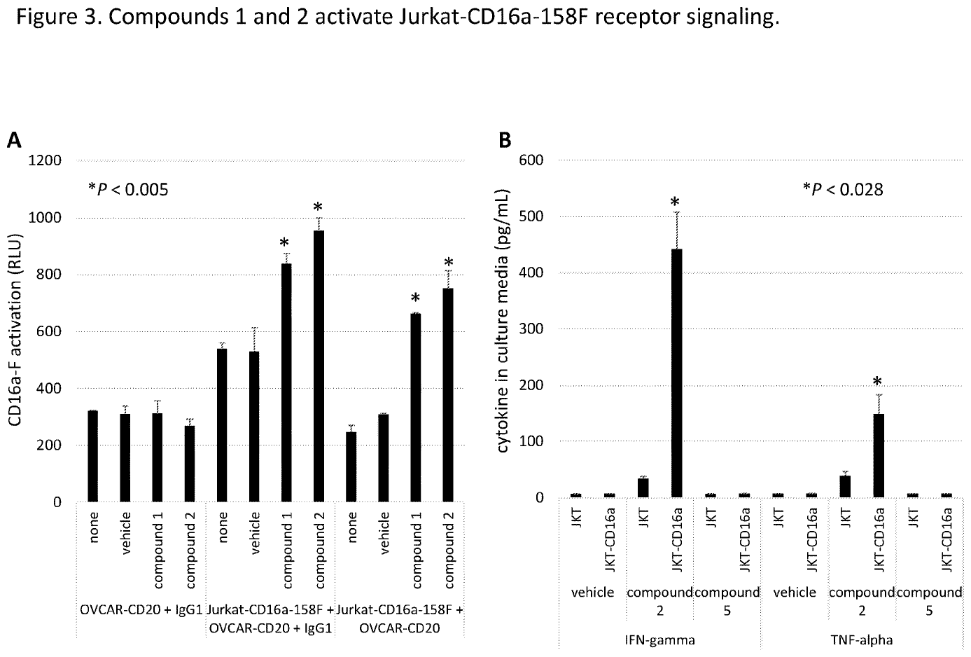

To confirm that the enhanced CD16a FcR-IgG antibody binding had biological relevance, bioassays were established using the Jurkat-CD16a-158F-luciferase (JKT-CD16a) reporter cell line (Promega) to test for the ability of Compound 1 and 2 to enhance and activate the low affinity CD16a-158F FcR in the presence or absence of IgG1 plus: target cells; target cells only; or JKT-CD16a cells only. The target cells used were OV-CD20, a human ovarian cancer cell line (OVCAR3) that is stably transduced to express the human CD20 antigen. The test antibody used was the chimeric (mouse variable region fused to the human IgG1 Fc domain) anti-CD20 rituximab. Briefly, 0 to 5,000 target cells were seeded into black 96-well microplates in RPMI with 7.5% fetal bovine serum (FBS) and 1% L-glutamine growth media (R7.5), and grown overnight at 37° C. in 5% CO₂. The next day, wells were washed and plated with 50 uLs of assay media (clear RPMI, 1% low IgG FBS and L-glutamine (R1)) containing varying concentrations of Compound 1 or 2 with or without 2.5 μg/mL of the test rituximab antibody that has been previously shown to specifically engage with OV-CD20 cells (Grasso L, et al. Oncol Letters 23:1-10, 2022). Finally, 9,400 JKT-CD16a cells in R1 media were added to each well and plates were incubated for 12-18 hours at 37° C. in 5% CO₂. Wells were tested for CD16a FcR activation using BIO-GLO (Promega) luciferase substrate following the manufacturer's recommendation and analyzed for relative luciferase production (RLU) using a multi-well plate reader (Varioskan). As shown in FIG. 3A, both Compounds 1 and 2 showed significant enhancement of JKT-CD16a activation in the presence of IgG (middle set of bars) in contrast to vehicle or no compound control wells. JKT-CD16a cells plus target cells without antibody were also tested for CD16a-158F FcR activation using similar methods as above. As shown in the right-side group of bars in panel A, both Compounds 1 and 2 stimulated JKT-CD16a-158F FcR activation in the absence of IgG antibody demonstrating the ability of these compounds to directly stimulate CD16a FcR activation, however at lower levels than in the presence of IgG antibody. Wells in which no JKT-CD16a cells were added (left-side bars) showed no enhancement in CD16a activation under any treatment condition. To confirm CD16a FcR specificity by Compound 1 and 2, parental Jurkat cells, which do not express CD16a FcR, and JKT-CD16a cells were tested for the production of the downstream CD16a FcR activation markers IL-1β and TNFα in culture media by ELISA. Opaque 96-well microplates were seeded with 50,000 cells/well in R1 media and 10 μM of Compound 2 or its inactive Compound 5 analog were added to each well and incubated for 18 hours at 37° C. in 5% CO₂. Plates were centrifuged to pellet cells and supernatants collected and tested for INFγ (Sino Biologicals) and TNFα (R&D Systems) production via ELISA following the manufacturer's recommendations. As shown in FIG. 3B, both cytokines were produced by JKT-CD16a-158F cells when grown in the presence of Compound 2 while parental Jurkat or any cells treated with Compound 5 had little to no cytokine production, thus demonstrating the need for the CD16a FcR for induced cytokine production by Compound 2. These data demonstrate the use of compounds and similar analogs to be able to: 1) enhance the CD16a-158F FcR binding to IgG1 antibodies and 2) stimulate CD16a FcR activation in the absence of antibody binding, both features useful for the pharmaceutical development of CD16a FcR based therapies. Experiments represent a minimum of triplicate wells. P values were generated comparing compound treated vs vehicle for each group using the Student's T-test.

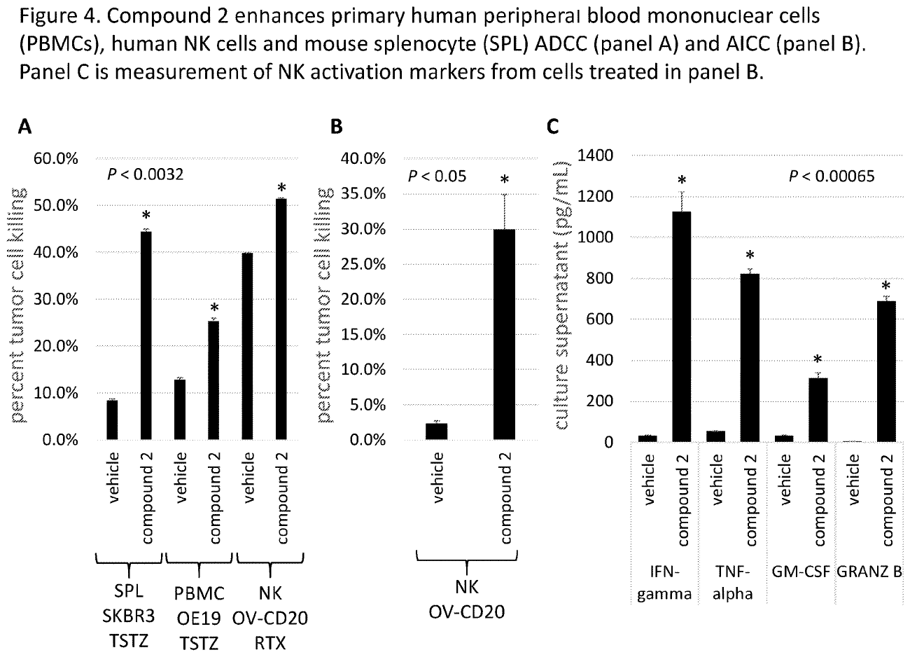

To demonstrate the ability of active compounds such as Compound 2 to stimulate the immune-effector activity of primary immune cells, human peripheral blood mononuclear cells (PBMC), human NK and mouse splenocyte (SPL) effector cells were tested for activation by Compound 2 by measuring antibody dependent (ADCC) or antibody independent (AICC) cellular cytotoxicity against tumor target cells. Briefly, 5,000 human breast carcinoma (SKBR3 or OE19) or OV-CD20 target cells were plated in black 96-well microplates in R7.5 media for 48 hours. Wells were washed and 50,000 PBMC, NK or SPL effector cells were added in R1 assay media with or without a targeting test antibody and 25 or 50 μM of Compound 2. Vehicle is the solvent that Compound 2 is dissolved in. For assays employing SKBR3 human breast cancer or OE19 esophageal adenocarcinoma cell lines, 2.5 μg/mL of the HER2-targeting trastuzumab test antibody was used for ADCC assays. For OV-CD20 assays, 2.5 μg/mL of the CD20-targeting rituximab test antibody was used for ADCC assays. Cultures were incubated for 24 hours at 37° C. in 5% CO₂ in triplicate. After incubation, culture supernatants were harvested for cytokine analysis and wells were washed with phosphate buffered saline and adhered target cell viability was quantified via Cell Titer-GLO (Promega) using a Varioskan plate reader. As shown in FIG. 4A, wells treated with Compound 2 plus targeting antibody demonstrated that Compound 2 could enhance human (PBMC and NK cells) and mouse (SPL cells) effector cell killing of target cells through ADCC as compared to antibody treatment alone (vehicle). ADCC data was graphed as percent killing of antibody treated with Compound 2 or vehicle vs. cultures not treated with antibody. Using similar methods as above, FIG. 4B shows the ability of Compound 2 to activate human primary NK cells in the absence of antibody that in turn enabled NK cell killing of OV-CD20 tumor cells via AICC mechanisms. AICC results were graphed as percent killing by Compound 2 vs vehicle only treated cultures. To demonstrate that CD16a FcR pathway activation in NK cells were involved in the AICC effect, supernatants from Compound 2 and vehicle treated wells from experiments in FIG. 2B were analyzed for cytokines and granzymes associated with CD16a FcR mediated effector cell activation via ELISA using anti-cytokine antibody combinations recommended by the respective manufacturers. As shown, TNFα, IFNγ and GM-CSF (Abcam) were all found to be produced and secreted by NK cells in Compound 2-treated wells in the absence of antibody in addition to the production of the cytotoxic granzyme B protein (Sino Biologics), supporting the evidence of CD16a FcR effector-mediated cell activation by Compound 2 and subsequent target cell killing (Wang R, et al. J Leukoc Biol 91:299-309, 2012; Louis C, et al. J Exp Med 217: e20191421; Caligiuri M A. Blood 112:461-469, 2008). Time course analysis found that activation and cytokine production occurred within 30 minutes of exposure. Similar cytokine production was found in Compound 2 treated human PBMC and mouse SPL cultures. These data demonstrate the ability of Compound 2 and active analogs to be able to enhance antibody-based target cell killing through antibody as well as non-antibody-based stimulation of human and murine immune-effector cells and their potential as therapies in CD16a FcR targeting diseases. Experiments represent a minimum of triplicate wells. P values were generated using the Student's T-test.

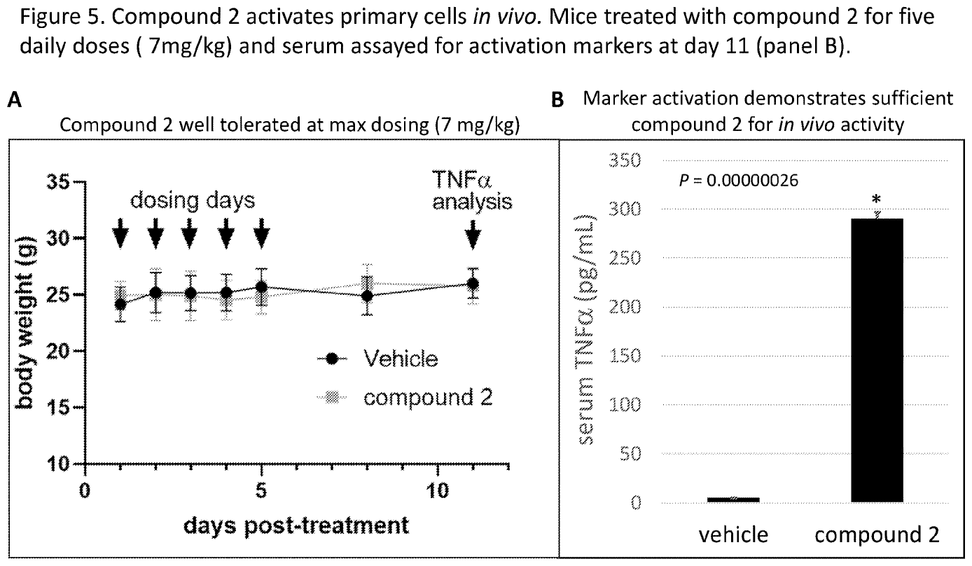

To demonstrate the ability of active compounds such as Compound 2 to activate immune-effector cells in vivo, we employed the use of athymic nude mice to determine tolerability and systemic immune-effector cell activation. Previous in vivo testing found that Compound 2 was tolerable up to at least 7 mg/kg of daily i.v. dosing in athymic nude mice. To determine the ability of Compound 2 to activate immune-effector cells in vivo, we administered 7 mg/kg of Compound 2 or similar volumes of vehicle (ethanol) daily for 5 days (day 1-5) and isolated total serum on day 11 to measure for immune-effector cell activation markers. As shown in FIG. 5A, the 5-day treatment was well tolerated by all mice in each cohort as determined by lack of body weight loss. The TNFα (R&D Systems) immune-effector cell activation marker was monitored in serum from treated mice via ELISA using similar methods as described above and found to be significantly upregulated in the Compound 2 treated mice (298 μg/mL) in contrast to the vehicle control cohort (11 μg/mL) P=0.00000026. These data demonstrate the ability of Compound 2, and its active analogs to activate immune-effector cells in vivo for evaluating potential therapeutic applications in mouse models of human disease and as potential therapies to treat CD16a FcR involved human diseases, such as cancer or infectious diseases with or without therapeutic antibody-based therapy. Experiments represent a minimum of triplicate wells. P values were generated using the Student's T-test.

### Example 2—Identification of Compounds Capable of Suppressing Viral Killing of Human Perpheral Blood Mononuclear Cells

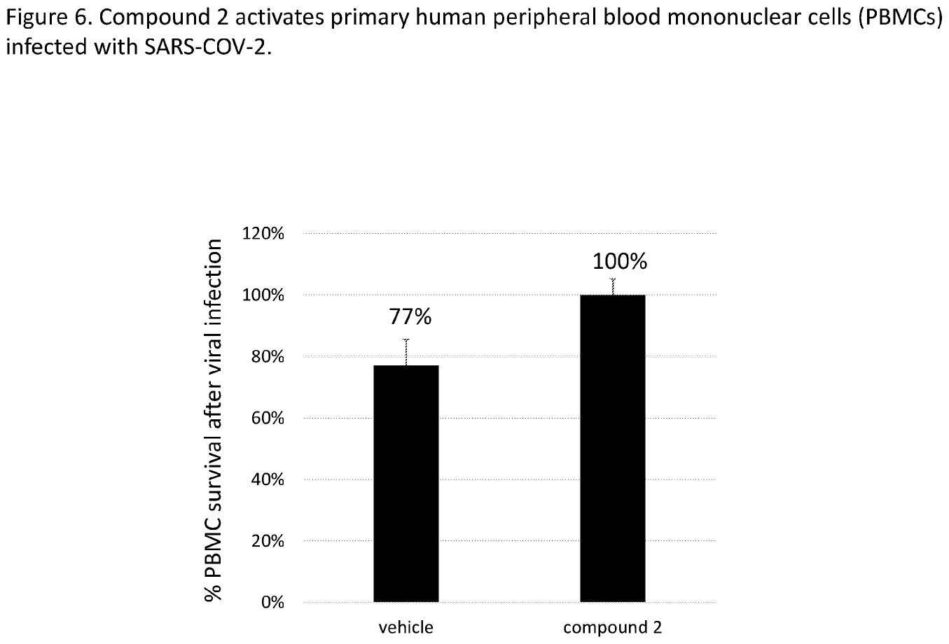

NK cells have been previously reported to be capable of killing virally infected cells (Bjorkstrom N K, et al. Nat Reviews Immunol 22:112-123, 2022). In particular, previous studies have shown that the SARS-COV-2 virus can infect human PBMCs and this infection can be lethal to overall PBMC survival (Manunta MDI, et al. Scientific Reports 11:4904-4916, 2021). To test the ability of CD16a FcR activating small molecular weight agents described here to activate effector cells within PBMCs that in turn can kill virally infected PBMC sib cells and reduce viral load, Compound 2 was tested against human PBMCs exposed to SARS-COV-2 virus. Briefly, IL2-activated PBMCs grown in RPMI media with 10% heat inactivated FBS (R10) were plated at 100,000 cells/well in 96 well U-bottom microplates. Next, 2.7×105 viral titers (previously determined by plaque forming units, PFU) were added to appropriate wells with or without 25 μM of Compound 2. Cultures were incubated for 72 hours at 37° C. in 5% CO₂. Plates were centrifuged and supernatants collected for future cytokine and viral titer analysis. Cells were resuspended in RPMI plus 10% FBS, transferred to 96-well black microplates and tested for viability using Cell Titer-Glo (Promega) and read for viability on a 96-well microplate reader. As shown in FIG. 6, PBMC cultures treated with virus and vehicle had 77% viability in contrast to those treated with 25 μM of Compound 2 that retained 100% viability. These data support a role for Compound 2 and related analogs for treating virally infected cells and patients afflicted with infectious disease. Experiments represent a minimum of triplicate wells.

### Example 3—Screening for Active Analogs and Defining Key Regions for Maintaining Enhanced Cd16A Fc Receptor-Antibody Binding and Immune-Effector Cell Activation

The use of the molecular-based recombinant CD16a-HIS FcR and IgG1 antibody ELISA assays enable facilitated screening to identify optimal regions within Compounds 1 and 2 as well as the related 3-cyclohexyl,1-cyclopentyl related bisnoralcohol (Compound 3 and 4) in addition to new analogs that may further enhance or suppress the specific activation of CD16a FcR and downstream immune-effector cell activities. Leads identified by the molecular screens taught here may then be tested in in vitro bioassays for confirmation and then in vivo for identifying the most pharmacokinetically, pharmacodynamically, and therapeutically optimal as well as best tolerated compound(s). Here, we first tested a number of compounds in the public domain that have common structures to Compound 1 or 2. As shown in FIG. 7A, these studies demonstrate the importance of the hydroxyl or formyl group as R1 where the highly similar Compound 2 analog, cycloartenol ferulate (Compound 12, FIG. 7B), which lacks the hydroxyl group at R1, is completely inactive while the 3-formyl cycloartenol 24-ketone analog containing a formyl group at R1 (Compound 20) retains enhanced CD16a FcR-IgG antibody binding as does the 3-β-hydroxy-4,4-dimethyl-5-a-bisnoralcohol analog (Compound 4) in contrast to the 3-α-hydroxy-4,4-dimethyl-5-a-bisnoralcohol (Compound 17), again showing importance of stereospecificity at the R1 region.

In addition, the importance of methyl groups at R2 and R3 for CD16a FcR-IgG antibody enhanced binding and activation is shown in the loss of activity by 5α-cholesta-8,24-dien-3β-ol (Compound 6) as compared to Compound 1 while stereospecificity at C13 and C14 has some negative impact on activity as shown by Compound 13. The exchange of a methyl group with fluorine at R4 in Compound 1, appears to have little impact on activity.

Synthesis of analogs using the cholestane or Compound 3 backbone based on the findings above further demonstrated the flexibility of making diverse analogs at the R5 and R6 regions, whereby PEG dimerization of two cholestane-derived 9β-19-cyclo-24-lanosten-3β-ols at the R6 region (Compound 14) or substitution of a ketone at C24 on cholestane-derived Compound 20 demonstrate little to no loss of enhanced CD16a binding activity, similar to the retained binding activity of the Compound 3 derived Compound 4, leading a focused effort to further expand structural activity relationships of varying chemical classes for further optimization in the R5/R6 region. These as well as additional structure activity relationships found in the Compounds 1 through 20, Compound 30 and Compounds 36 through 38 provide additional teachings of active regions to generate additional active analogs for the treatment of CD16a FcR involved disease indications, including cancer and infectious diseases. Alternatively, non-enhancing compounds such as compound 37 or 38 may be used as inhibitors of CD16a activation to treat CD16a-mediated inflammatory diseases.

Based on the finding that active Compound 1, 2, 3 or 4 derived analogs have extreme variability at R5 and R6 regions, additional examples of modifications can be implemented to improve CD16a FcR-IgG antibody binding and/or activation as well as to be used as biotinylated probes (Compound 34, FIG. 8) to analyze compound-CD16a FcR interactions in vitro and in vivo (FIG. 8.). These modifications are useful to improve solubility, molecular activity, PK/PD qualities and therapeutic activity. Methods for synthesis for other similar types of compound structures are provided.

A general scheme was implemented to produce additional analogs of Compound 2 varying at R5/R6 and accommodate the necessary requirements to enhance CD16a-FCR binding to IgG1 antibody as well as FcR activation of CD16a expressing effector cells for therapeutic applications. To prepare these compounds, Compound 12 was generated and used as a source to generate Compound 2 and its analogs. In scheme 1, base hydrolysis, protection of the 3-hydroxyl group, cleavage of the isopropylidine, followed by the removal of the 3-hydroxyl protecting group yields Compound 21 that was then used as an active analog and a common intermediate to create additional Compound 2 based R5/R6 analogs via: reductive amination at C24 (scheme 2); oxidation to generate carboxylic acid analogs (scheme 3); and acetylation and sulfation of intermediates (scheme 4).

In scheme 1 below, Compound 2 is generated by hydrolyzing Compound 12 in the presence of aqueous potassium hydroxide (KOH) in ethanol (EtOH). The resulting secondary alcohol is protected by acetylation using acetic anhydride. The double bond was then oxidized using O3, Zn and AcOH or potassium osmate and sodium periodate to generate the aldehyde 1-2, followed by hydrolysis in the aqueous 80% NaOH/EtOH to create Compound 21. As Compound 12 is derived from natural sources, previous experiences have found that a C24 ketone isomer is consistently present, which enables the generation of Compound 30 (FIG. 8), using the same method, but purified by supercritical fluid chromatography (SFC) and confirmed by nuclear magnetic resonance (NMR).

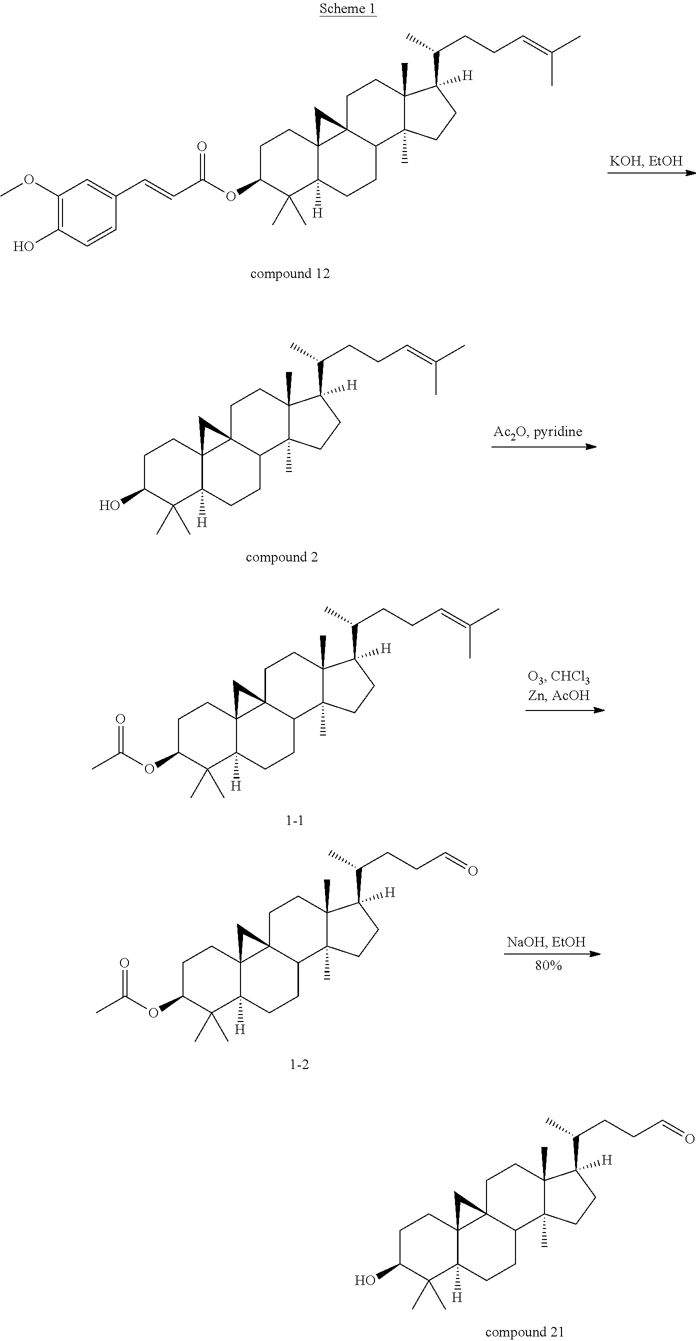

In scheme 2 below, Compound 25 is prepared using reductive amination in MeOH and/or DCM from Compound 21 and commercial dimethylamine hydrochloride.

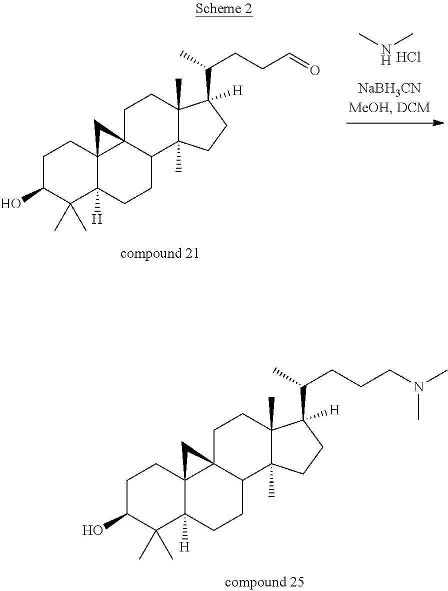

Another analog generated via scheme 2 is Compound 26 that is prepared using reductive amination in MeOH and DCM from Compound 21 and commercial isopropylamine.

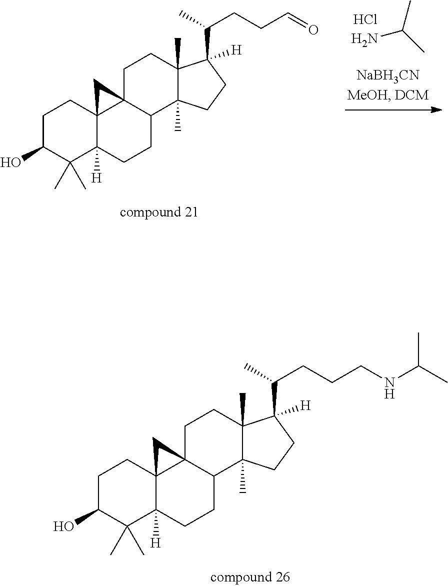

Compound 27 is prepared via scheme 2 using reductive amination in MeOH and DCM from Compound 21 and commercial piperidine

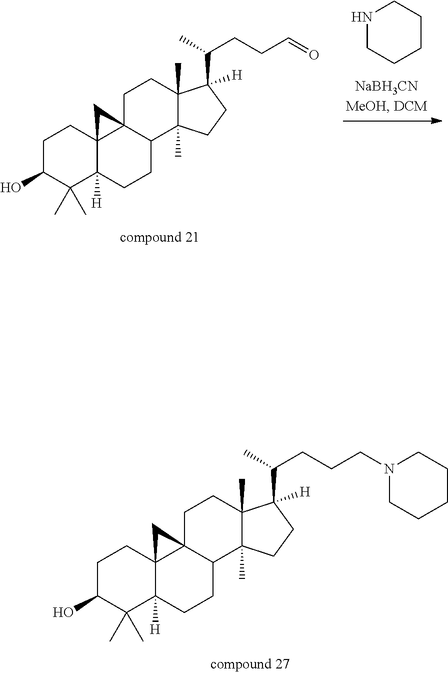 [SMILES: C[C@H](CCC=O)[C@H]1CC[C@@]2(C)C3CC[C@H]4C(C)(C)[C@@H](O)CC[C@@]45C[C@@]35CC[C@]12C ; C1CCNCC1 ; C[C@H](CCCN1CCCCC1)[C@H]1CC[C@@]2(C)C3CC[C@H]4C(C)(C)[C@@H](O)CC[C@@]45C[C@@]35CC[C@]12C]

Compound 2 analog 28 generated via scheme 2 methods is prepared using reductive amination of Compound 21 in MeOH and DCM and commercial morpholine.

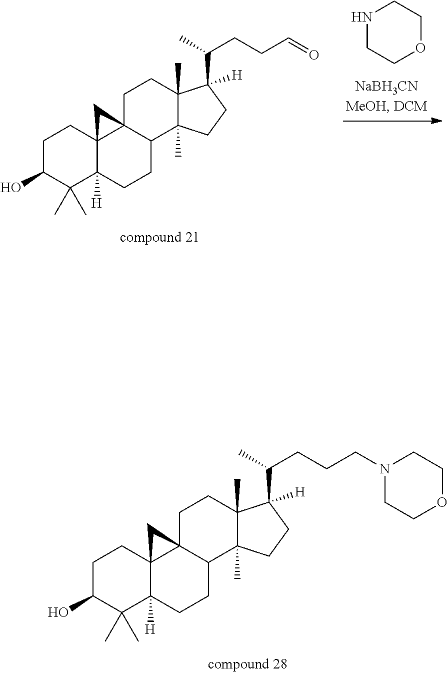 [SMILES: C[C@H](CCC=O)[C@H]1CC[C@@]2(C)C3CC[C@H]4C(C)(C)[C@@H](O)CC[C@@]45C[C@@]35CC[C@]12C ; C1COCCN1 ; C[C@H](CCCN1CCOCC1)[C@H]1CC[C@@]2(C)C3CC[C@H]4C(C)(C)[C@@H](O)CC[C@@]45C[C@@]35CC[C@]12C]

In another scheme 2 synthesis, Compound 29 is generated by oxidization of Compound 21 in the presence of NaClO₄, NaH₂PO₄ and 2-methyl-2-butene in t-BuOH and THE to produce Compound 29.

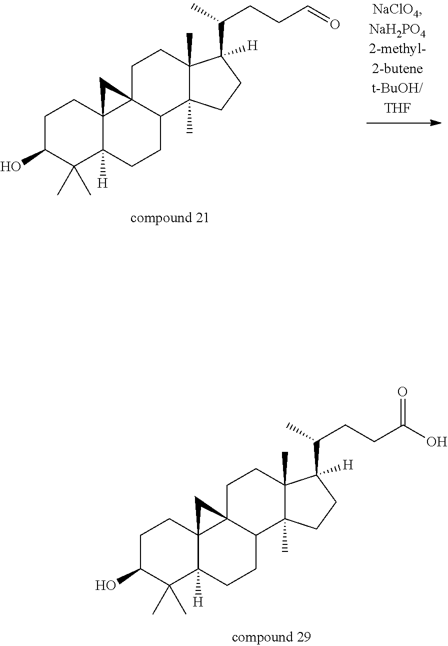 [SMILES: C[C@H](CCC=O)[C@H]1CC[C@@]2(C)C3CC[C@H]4C(C)(C)[C@@H](O)CC[C@@]45C[C@@]35CC[C@]12C ; C[C@H](CCC(=O)O)[C@H]1CC[C@@]2(C)C3CC[C@H]4C(C)(C)[C@@H](O)CC[C@@]45C[C@@]35CC[C@]12C]

And as a last example for scheme 2 analog synthesis, pegylated R6 analogs of Compound 2 with varying lengths of 2 to 12 polyethylene glycol (PEG) units are synthesized using reductive amination in MeOH and DCM from the Compound 21 and commercial NH₂-PEG2-OH (Compound 32), NH₂-PEG12-OH (33) and PEG lengths in between.

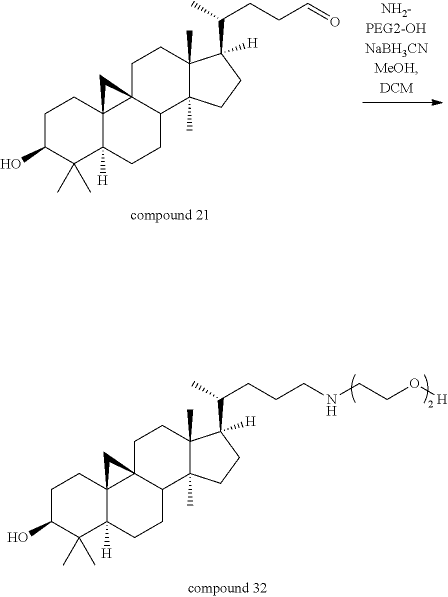

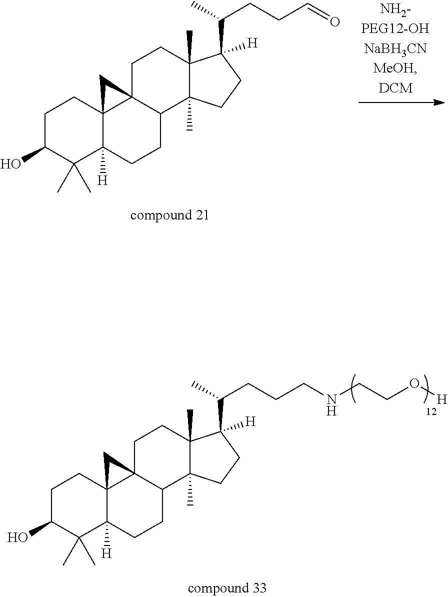

In scheme 3 below, Compound 22 is prepared using reductive amination in MeOH and DCM from the Compound 21 and commercial BnNH₂, followed by hydrogenation in the presence of Pd(OH)₂/C, H₂ atmosphere and MeOH. The Compound 22 is then sulfonylated by methyl sulfonyl chloride under basic condition to yield Compound 23, while Compound 24 is prepared from Compound 22 by condensation and acetic acid in the presence of EDCI and DMAP in DCM.

### Scheme 3 and 4

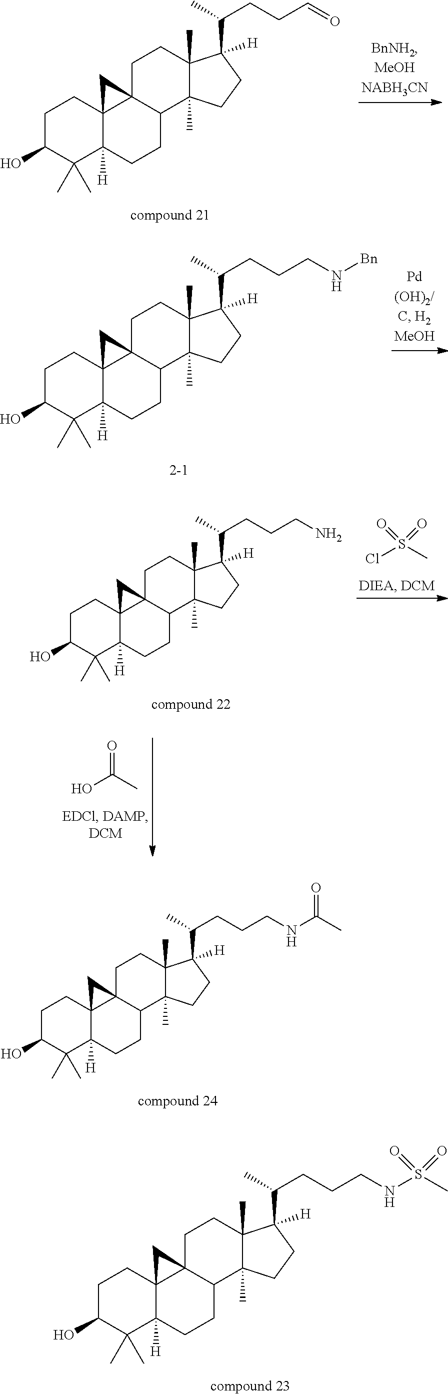

In another method, Compound 21 is reduced in the presence of NaBH4 in EtOH to yield Compound 31, which is then condensed with D-(+)-Biotin in the presence of EDCI and DMAP in DCM to yield biotinylated Compound 34.

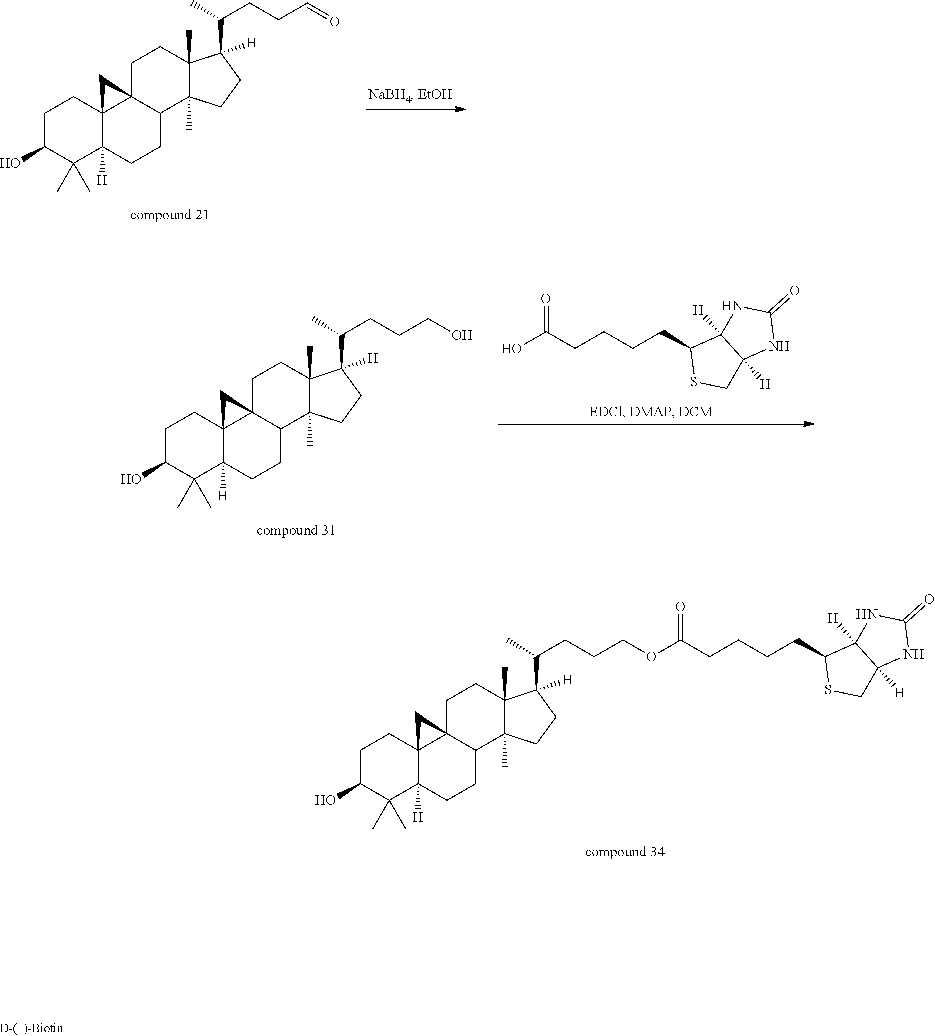 [SMILES: C[C@H](CCC=O)[C@H]1CC[C@@]2(C)C3CC[C@H]4C(C)(C)[C@@H](O)CC[C@@]45C[C@@]35CC[C@]12C ; C[C@H](CCCO)[C@H]1CC[C@@]2(C)C3CC[C@H]4C(C)(C)[C@@H](O)CC[C@@]45C[C@@]35CC[C@]12C ; O=C(O)CCCC[C@@H]1SC[C@@H]2NC(=O)N[C@@H]21 ; C[C@H](CCCOC(=O)CCCC[C@@H]1SC[C@@H]2NC(=O)N[C@@H]21)[C@H]1CC[C@@]2(C)C3CC[C@H]4C(C)(C)[C@@H](O)CC[C@@]45C[C@@]35CC[C@]12C]

Similar methods were used to generate compound 1 analogs with similar R5 and R6 modifications.

### Example 4—Identification of Compounds with Optimal Pharmacologic Properties for In Vivo Use

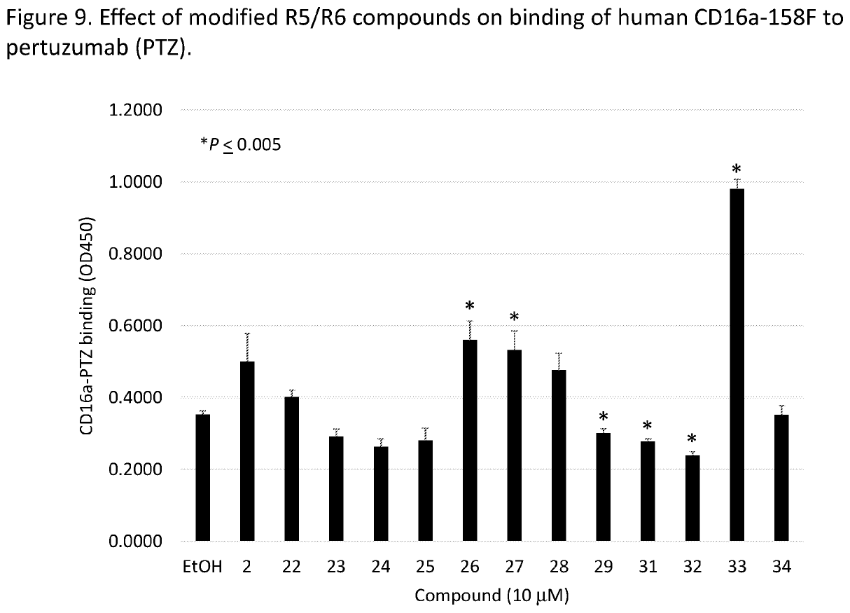

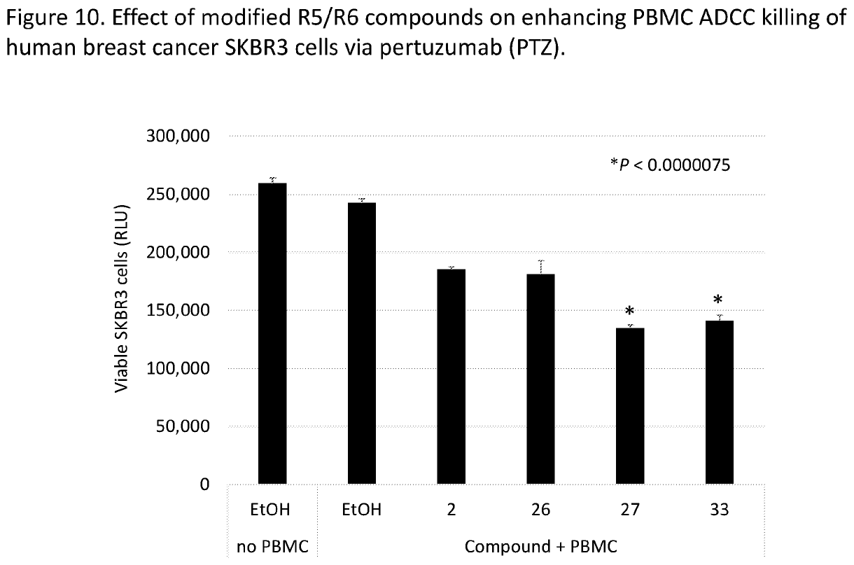

Analysis of Compound 2 derived analogs described in Example 3 were conducted testing for enhanced CD16a FcR binding to IgG via ELISA. Briefly, 96 well plates were coated with PTZ as described above and probed with CD16a-158F-HIS in the presence of 10 μM parental Compound 2 and R5/R6 Compound analogs 22-34 to assay for those mediating enhanced CD16a-IgG binding. As shown in FIG. 9, compounds 26, 27 and 33 significantly enhanced CD16a-158F binding to IgG (P<0.005). To further evaluate the impact of enhanced binding these compounds were tested for enhancing human PBMC ADCC as described above. Human breast cancer SKBR3 cells were plated in black 96-well microplates at 5,000 cell/well in R7.5 and grown overnight at 37° C. in 5% CO₂. The next day 50,000 PBMCs in R1 medium were added per well in the presence of 1 or 5 μM of each compound or ethanol (EtOH) vehicle control in triplicates. Wells treated with EtOH and PTZ without PBMCs were used as a negative control. Cultures were incubated for 24 hours at 37° C. in 5% CO₂ in triplicate. After incubation, target cell viability was quantified via Cell Titer-GLO (Promega) using a Varioskan plate reader. Units are shown as relative luminescent units (RLU). As shown in FIG. 10, Compounds 27 and 33 enhanced ADCC most significantly (P<0.0000075).

We next tested Compounds 2, 26, 27 and 33 for metabolic stability using human and mouse microsomes. For each test compound, samples were diluted a final concentration of 2 μg/mL in 25 mM potassium phosphate buffer with liver microsomes of each species at a final concentration of 0.5 mg/mL. The enzyme reaction was initiated by the addition of NADPH reagent at a final concentration of 1 mM. For negative control samples, NADPH reagent was not added. Samples were incubated at 37° C. on a 50-RPM orbital shaker, and an aliquot was removed at pre-determined timepoints at 0, 5, 10, 15, 30, and 60 minutes. Samples were then precipitated with three volume of acetonitrile containing propranolol as an internal standard, and centrifuged for 10 min at 2000 g then analyzed by LC/MS/MS. Note that compounds undergo only Phase I metabolism in liver microsomes in the presence of NADPH cofactors. Data interpretation of results are based on percent parent compound remaining relative to 0-minute incubation samples, from which the elimination half-life is calculated based on the natural log of % compound remaining vs. time plot. The following parameters were calculated to estimate the compound's in vitro metabolic stability: Cmp=concentration of microsomal proteins (mg/mL); t_{1/2}=the half-life (min), where t_{1/2} is equal to 0.693/slope; CLint=the intrinsic hepatic clearance (μL/min/mg), where CLint is equal to 0.693/(t_{1/2}×Cmp). Based on these analyses, Compounds 26 and 27 had significantly improved metabolic stability in both mouse and human microsomes in contrast to Compounds 2 and 33 as shown in Table 1.

TABLE 1
Metabolic Profiles of Compounds 2,
26, 27 and 33 in Liver Microsomes

| | T½ (minutes) | | CLint (μL/min/mg protein) | |
|---|---|---|---|---|
| | mouse | human | mouse | human |
| compound 2 | 113 | 70.5 | 12.3 | 19.7 |
| compound 26 | 641 | 456 | 2.16 | 3.04 |
| compound 27 | 610 | 701 | 2.27 | 1.98 |
| compound 33 | 65.8 | 64.5 | 21.1 | 21.5 |

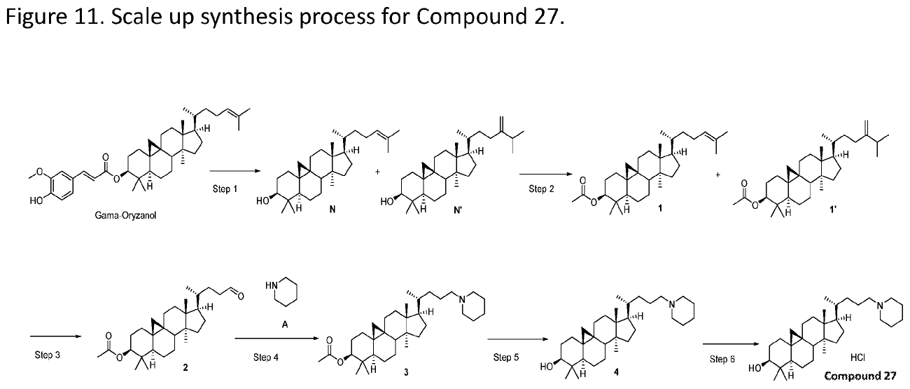

Based on the superior metabolic stability (Table 1) and ADCC enhancement of Compound 27 by PBMCs (FIG. 10), we further analyzed the compound for in vivo activities. We first scaled up of the synthesis of Compound 27 as described below and depicted in FIG. 11. A 25 gram batch of Gama-Oryzanol was hydrolyzed under basic condition of aqueous 0.415M KOH in EtOH to provide the secondary alcohol N. The secondary alcohol N was then protected with acetyl group using 37.8 mM of Ac₂O, followed by ozonolysis to provide the aldehyde intermediate 2. A subsequent reductive amination was carried out using 27.1 mM piperidine in AcOH, followed by addition of NaBH(OAc)₃ to provide an intermediate 3. The freebase of Compound 27 was obtained by hydrolysis of the intermediate 3 under the condition of aqueous NaOH in MeOH and THF. The freebase of Compound 27 was converted to its HCl salt using 4M HCl dioxane solution and the precipitated product was collected by filtration. The final product was analyzed by mass spectrometry and H-NMR. Mass spectrum analysis showed a predominant compound with expected Compound 27 molecular weight of 470.6 (see below) and an accurate H-NMR spectral profile.

Mass spectrum analysis of Compound 27 revealed a major species having a molecular weight of 470.6, as predicted for Compound 27.

To further determine the pharmacologic properties of Compound 27, we tested for its oral bioavailability and ability to activate NK cells systemically in mice as determined measuring NK cell activation markers IFNγ and TNFα. For oral bioavailability, mice were fed a standard laboratory rodent diet and housed in individual cages on a 12-hour light and 12-hour dark cycle with at 22±30° C. and relative humidity at 50±20%. Animals are fasted overnight before dosing, with food returned after 6 hours at which time blood samples are obtained. Water is provided ad libitum throughout the study. Compound 27 was prepared by grinding its precipitated powder into fine particles and suspended in 0.5% hydroxypropyl methylcellulose (HPMC) in water. Three animals were dosed via gavage needle for oral administration at 100 mg/kg (10 mL/kg). Blood samples (25-30 μL per sample) were taken via the saphenous vein at 5, 15, 30 minutes, and 1, 2, 4, 6, 8, 24 hours after administration. Additional blood samples (150-200 μL per sample) were taken at 24 hours for cytokine analysis. Blood samples were collected in Greiner MiniCollect K₂EDTA tubes, placed on ice, and within 30 minutes, centrifuged at 15,000 g for 5 min to obtain plasma samples with additional 50-100 μLs of each plasma sample collected at 24 hours. All plasma samples were stored at −70° C. until analysis.

Bioanalysis of plasma samples were conducted as follows. Plasma samples were prepared using three volumes of acetonitrile containing internal standard to one volume of plasma to precipitate proteins. Samples were centrifuged at 3000 g for 10 minutes and supernatant removed for analysis by LC-MS/MS. Calibration standards and quality controls were made by preparation of a 1 mg/mL stock solution and subsequently a series of working solutions in methanol:water (1:1, v/v), which were spiked into blank plasma to yield a series of calibration standard samples ranging from 1 ng/ml to 10 μg/mL and quality control samples at three concentration levels (low, middle and high). All incurred plasma samples were treated identically to the calibration standards and quality control samples. LC-MS/MS analysis was performed utilizing multiple reaction monitoring for detection of characteristic ions for each drug candidate, additional related analytes and internal standard. Plasma concentrations are measured as described above to determine a concentration vs. time profile. The area under the plasma concentration vs time curve (AUC) is calculated using the linear trapezoidal method, known by those skilled in the art. Fitting of the data to obtain pharmacokinetic (PK) parameters was carried out using non-compartmental analysis.

Key PK parameters reported following intravenous administration are as follows: terminal half-life t_{1/2}, initial plasma concentration C₀, area under the plasma concentration vs. time curve AUC, volume of distribution at steady-state Vss, total plasma clearance CLp, and mean residence time MRT.

Key PK parameters reported following extravascular administration are as follows: terminal half-life t_{1/2}, maximum plasma concentration Cmax, time to reach maximum plasma concentration tmax, area under the plasma concentration vs. time curve AUC, mean residence time MRT, and bioavailability F. All parameters are expressed for individual animals as well as mean, standard deviation, and coefficient of variation and are commonly known by those skilled in the art.

Following oral administration at 100 mg/kg in suspension, the test compound gradually reached a peak plasma concentration (Cmax) of 511 ng/mL within 6 hours (Tmax) and its plasma concentration declined in a multiphasic manner with a last measurable concentration of 242 ng/mL at 24 hours and a long terminal half-life (t_{1/2}) of 18.3 hours. The total systemic exposure (AUClast) was 7.75 h*μg/mL with an oral bioavailability (F) of 11.7%. These data demonstrate the oral bioavailability of Compound 27.

To determine the tolerability and ability to systemically activate NK cells in mice, we tested orally administered Compound 27 to mice. Six mice were randomized into treatment groups vehicle control (water) and 100 mg/kg Compound 27 all suspended in 0.5% hydroxypropyl methylcellulose. Mice were dosed orally on days 1-5, 7, 9, 11, 13, 15 and sacrificed on day 16 at which time plasma was collected. Tolerability was based on Day 1 bodyweight of each mouse. The maximum tolerated dose is determined as weight loss exceeding 20% or >10% of animals in a group die. After the 10 course dosing, all mice in both groups were found to be healthy with similar body weights, confirming that 100 mg/kg of multiple oral dosing of Compound 27 is well tolerated.

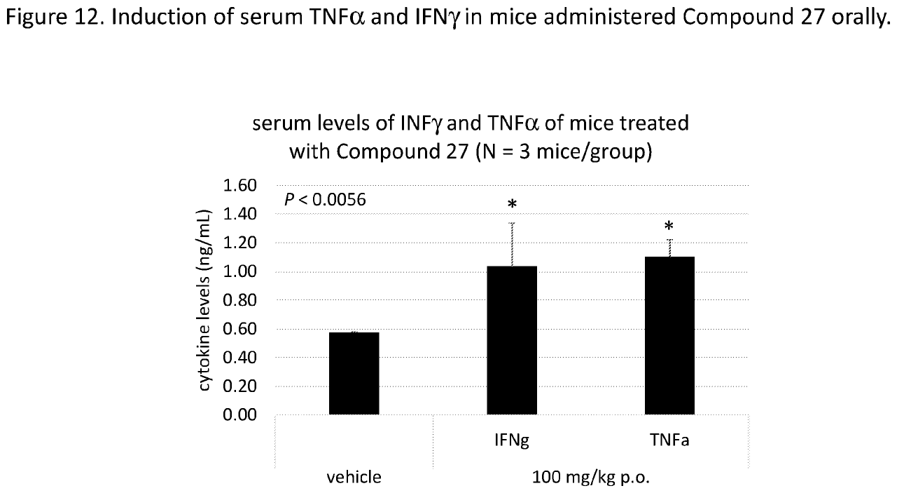

To confirm that oral administration of Compound 27 activated NK cells, the NK activation markers IFNγ and TNFα were measured from day 16 harvested plasma via ELISA using methods described in Example 1. As shown in FIG. 12, both NK cell activation markers were upregulated in Compound 27 treated mice in contrast to vehicle control treated mice, confirming oral uptake of Compound 27 and activation of target NK cells. These scaffolds embody a primary inventive step that is taught within this application.

### Example 5—Development of Analogs Using Compound 1, 2, 3 and/or 4 Scaffolds to Develop Cd16a Pathway Inhibitors to Suppress Inflammation

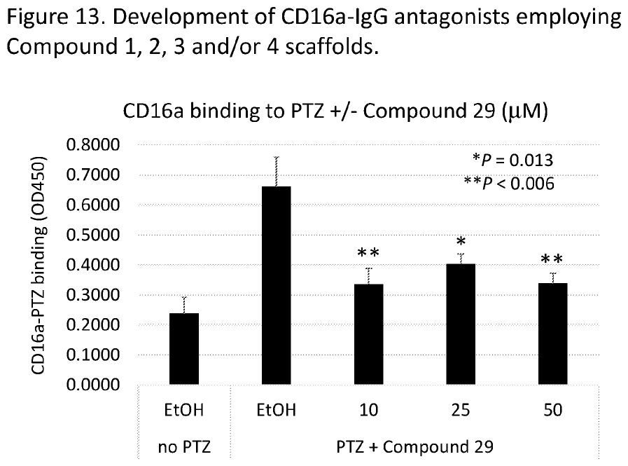

While many examples of analogs synthesized off of the Compound 1, 2, 3 and 4 scaffolds have shown enhanced activity of CD16a FcR binding and immune-effector cell activation, other analogs have been found to suppress IgG-C16a FcR binding. Analogs of this class may be useful for suppressing inflammatory diseases. Several reports have found that active CD16a signaling in inflammatory disease may be an underlying cause for a subset of inflammatory diseases (Steel A W, et al. Aliment Pharmacol Ther 33:115-126, 2011). The teaching here has shown that the Compound 1, 2, 3 and 4 scaffolds can be utilized to add chemical groups that can enhance their activity for CD16a stimulation. Here we provide an example of how these scaffolds can be used to develop CD16a antagonists to suppress CD16a-IgG interactions and downstream activities, including production of proinflammatory cytokines such as IFNγ. FIGS. 10 and 13 show ELISA binding assays as described in Example 1. Briefly, 96-well clear microplates were coated with 2.5 μg/mL pertuzumab (PTZ) IgG or HSA as negative control (no PTZ). Wells were then probed with human CD16a-158F-HIS with or without compounds to monitor CD16a-PTZ binding. As shown in FIGS. 10 and 13, multiple analogs derived from the Compound 2 scaffold (Compounds 29, 31, 32) were capable of suppressing CD16a-PTZ interaction by ~49% (P≤0.005) that in turn will suppress CD16a pathway signaling. The use of the scaffolds taught here can be expanded to develop even more potent CD16a inhibitors similar to the approach taught here to develop CD16a activators. These scaffolds embody a primary inventive step that is taught within this application.

The methods described within the examples provided here teach the art of new compositions useful for the treatment of CD16a FcR mediated diseases and therapies.

TABLE 1
SEQUENCE IDENTIFICATION (all sequences N to C terminal)

Underline denotes poly-histidine tag
BOLD denotes polymorphic change in human CD16a 158F and 158V
SEQ ID NO: 1 mature human CD16a-158F-HIS (accession number P08637)
GMRTEDLPKAVVFLEPQWYRVLEKDSVTLKCQGAYSPEDNSTQWFHNESLISSQASSYFIDA
ATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLFGSKNVSSETVNITITQGLAVSTISSFF
PPGGLNDIFEAQKIEWHEHHHHHH
SEQ ID NO: 2 mature human CD16a-158V-HIS (accession number P08637)
GMRTEDLPKAVVFLEPQWYRVLEKDSVTLKCQGAYSPEDNSTQWFHNESLISSQASSYFIDA
ATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFF
PPGGLNDIFEAQKIEWHEHHHHHH
SEQ ID NO: 3 mature human CD32a-131H-HIS (accession number P12318)
QAAAPPKAVLKLEPPWINVLQEDSVTLTCQGARSPESDSIQWFHNGNLIPTHTQPSYRFKANN
NSGEYTCQTGQTSLSDPVHLTVLSEWLVLQTPHLEFQEGETIMLRCHSWKDKPLVKVTFFQN
GKSKFSHLDPTFSIPQANHSHSGDYHCTGNIGYTLFSSKPVTITVQVPSMGSSSPMGIIVAVVIA
TAVAAVAAVVALIYCRKKRISANSTDPVKAAQFEPPGRQMIAIRKRQLEETNNDYETADGGY
MTLNPRADDDKNIYLTLPPNDHVNSNNGLNDIFEAQKIEWHEHHHHHH
SEQ ID NO: 4 mature mouse CD16a-1 (FCRG3)-HIS (accession number P08508)
ALPKAVVKLDPPWIQVLKEDMVTLMCEGTHNPGNSSTQWFHNGRSIRSQVQASYTFKATVN
DSGEYRCQMEQTRLSDPVDLGVISDWLLLQTPQRVFLEGETITLRCHSWRNKLLNRISFFHNE
KSVRYHHYKSNFSIPKANHSHSGDYYCKGSLGSTQHQSKPVTITVQDPATTGLNDIFEAQKIE
WHEHHHHHH
SEQ ID NO: 5 mature mouse CD16a-2 (FCRG4)-HIS (accession number 653142)
GLQKAVVNLDPKWVRVLEEDSVTLRCQGTFSPEDNSIKWFHNESLIPHQDANYVIQSARVKD
SGMYRCQTALSTISDPVQLEVHMGWLLLQTTKWLFQEGDPIHLRCHSWQNRPVRKVTYLQN
GKGKKYFHENSELLIPKATHNDSGSYFCRGLIGHNNKSSASFRISLGDPGSPSMFPPWHQGLN
DIFEAQKIEWHEHHHHHH

1. A method of treating a subject with a viral infection, a cancer, a parasitic infection, a bacterial infection, a yeast infection, or inflammatory disease, comprising:
administering an effective amount of Compound 1a, 1b, 2a, 2b, 4a, or 4b to the subject, whereby an immune response of the subject to the viral infection, the cancer, the parasitic infection, the bacterial infection, the yeast infection is increased, or whereby inflammation is decreased, wherein said compounds are represented as: 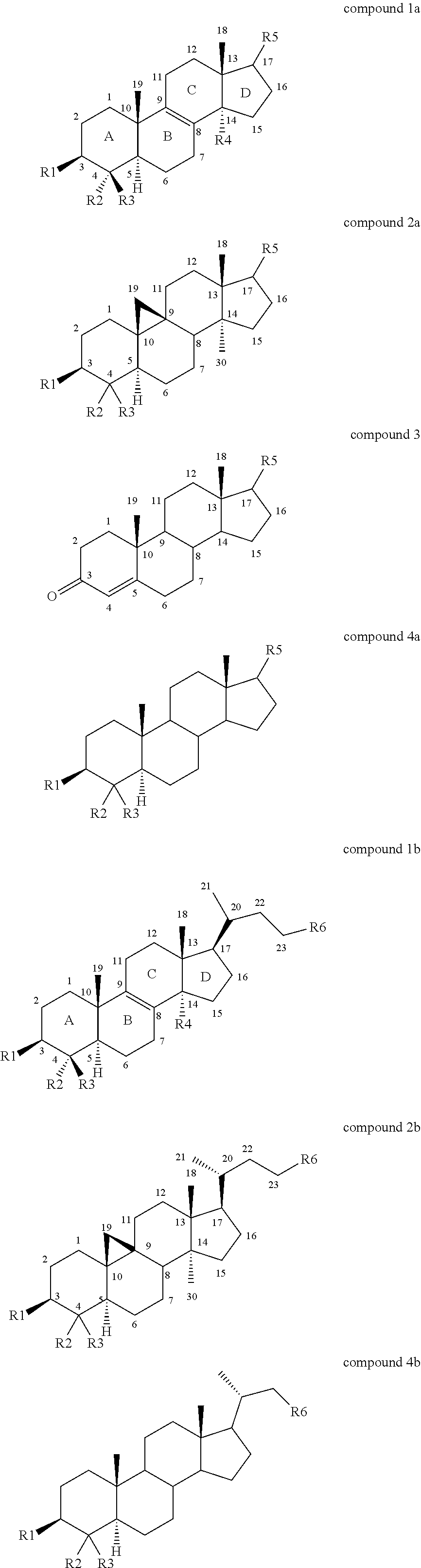
wherein R1 is hydroxyl (OH) or formyl (CH═O);
wherein R2 and R3 are methyl (CH₃);
wherein R4 is a methyl or 14a-difluoro-methyl (CHF₂);
wherein R5 and R6 are independently aldehyde (CH₃CHO); amine (NH₂);
dimethylamine ((CH₃)₂NH); piperidine ((CH₂)₅NH); isopropyl amine ((CH₃)₂CHNH₂); morpholine (O(CH₂CH₂)₂NH); carboxylic acid (C(═O) OH); sulfonamide (CH₃SO₂NH₂); acetamide (CH₃CONH₂) or (C2H₅NO); alcohol (OH); ketone (CH₃C(O)CH₃), isopropyl methyl ketone (CH₃COCH(CH₃)₂) or methyl-iso-butyl-ketone ((CH₃)₂CHCH₂COCH₃); methyl (CH₃); 2-methylheptane (CH₃CH(CH₂)₃CH(CH₃)₂); 2-methylheptane 2,3-diol (CH₃CH(CH₂)CH(OH)C(OH)(CH₃)₂); a pegylated amine comprising 2 to 12 PEG (polyethylene glycol) units; a PEG5 amine attached to Compound 1a, 1b, 2a, 2b, 4a, or 4b to form a dimer; and a biotinylated amine, wherein a biotin moiety is linked to an amine moiety by PEG 2-12.

2. The method of claim 1 further comprising administering a therapeutic antibody to the subject.

3. A method of assaying an antibody for binding to Fc receptor CD16a, comprising:
contacting the Fc receptor CD16a with Compound 1a, 1b, 2a, 2b, 4a, or 4b;
contacting the antibody with the Fc receptor CD16a; and
determining amount of binding of the antibody to the Fc receptor CD16a.

4. The method of claim 3, wherein the Fc receptor CD16a is on a cell surface.

5. The method of claim 3 wherein the Fc receptor CD16a is in a cell-free preparation.

6. A method of screening compounds for alteration of binding of an antibody to an Fc receptor CD16a, comprising:
contacting the Fc receptor CD16a with a compound;
contacting the antibody with the Fc receptor CD16a that has been contacted with the compound; and
determining a first amount of binding of the antibody to the Fc receptor CD16a that has been contacted with the compound; and
comparing the first amount of binding to a second amount of binding of the antibody to the Fc receptor CD16a that has not been contacted with the compound, wherein a compound that increases said amount of binding is a candidate therapeutic immunomodulator and a compound that decreases said amount of binding is a candidate therapeutic agent for inflammatory diseases.

7. The method of claim 6, wherein the Fc receptor CD16a is on a cell surface.

8. The method of claim 6 wherein the Fc receptor CD16a is in a cell-free preparation.

9. A method of screening compounds for activating or suppressing CD16a expressing cells, comprising:
culturing a sample of CD16a-expressing cells in the presence of a compound;
culturing a sample of CD16a-expressing cells in the absence of the compound;
determining amount of cell activation or suppression of the sample cultured in the presence and of the sample cultured in the absence of the compound, wherein a compound that increases cell activation therapeutic immunomodulator and a compound that decreases cell activation is a candidate therapeutic agent for inflammatory diseases.

10. The method of claim 9, wherein the CD16a-expressing cell is selected from the group consisting of a recombinant cell line expressing the CD16a receptor, a natural immune cell, a natural killer (NK) cell, a myeloid cell, a dendritic cell, and a monocytic cell.

11. A therapeutic antibody chemically linked to Compound 1a, 1b, 2a, 2b, 4a, or 4b.

12. A kit comprising:
Compound 1a, 1b, 2a, 2b, 4a, or 4b; and
a therapeutic antibody.

13. A composition for modulating an immune response in a human or rodent subject comprising:
Compound 1a, 1b, 2a, 2b, 4a, or 4b, wherein the compound is not lanosta-8,24-dien-3β-ol or 9β-19-cyclo-24-lanosten-3β-ol; and
a pharmaceutically acceptable carrier.

14. A nutritional supplement comprising Compound 1a, 1b, 2a, 2b, 4a, or 4b, wherein the concentration of the compound is greater than 700 mg/100 g of supplement.

15. The nutritional supplement of claim 14 wherein the compound is not lanosta-8,24-dien-3β-ol or 9β-19-cyclo-24-lanosten-3β-ol.

16. The nutritional supplement of claim 14 wherein the concentration of the compound is greater than 1 g/100 g of supplement.

17. The nutritional supplement of claim 14, which is a liquid beverage.

18. The nutritional supplement of claim 14, which is a solid snack bar.

19. A method of making an enriched food for increasing immune responses, comprising:
mixing a nutritional supplement comprising Compound 1a, 1b, 2a, 2b, 4a, or 4b with a food substance to form an enriched food suitable for increasing immune responses, wherein the compound is not lanosta-8,24-dien-3β-ol or 9β-19-cyclo-24-lanosten-3β-ol, wherein the concentration of the compound in the nutritional supplement is greater than 700 mg/100 g of supplement.

20. The method of claim 19 wherein the food substance is a fiber supplement.

21. A method of treating inflammation in a subject, comprising:
administering an effective amount of a compound to the subject, whereby an inflammatory response of the subject is decreased, wherein the compound is selected from the group consisting of bisnoralcohol, 5a-cholesta-8,24-dien-3β-ol, cholest-5,24-dien-3β-ol, 5a-cholest-7-en-3β-ol, 3β-chloro-cholest-5-ene, cycloartenyl ferulate, 3-hydroxy-4,4-dimethyl-5-ene-bisnoralcohol, 3-a-hydroxy-4,4-dimethyl-5-ene-bisnoralcohol, 2-methyl-3-β-hydroxy-4,4-dimethyl-5-ene-bisnoralcohol, 3B-lanost-8, 24,25-dihydroxy-en-3-ol, lanosta-8,24-dien-3β-ol-25-morpholino, and 9β-19-cyclo-24-lanosten-3β-ol-25-morpholino.

22. The method of claim 21 wherein the subject has an inflammatory disease or inflammatory syndrome.

23. The method of claim 21 wherein the inflammatory disease is selected from the group consisting of: Ankylosing Spondylitis (AS), Antiphospholipid Antibody Syndrome (APS), Gout, Inflammatory Arthritis, Myositis, Multiple Sclerosis, Rheumatoid Arthritis, Scleroderma, Sjogren's Syndrome, Systemic Lupus Erythematosus (SLE, Lupus), and Vasculitis.

24. A method of assaying a compound for suppression or enhanced activation or of Fc receptor CD16a on a cell surface, comprising:
contacting the Fc receptor CD16a on the cell surface with Compound 1a, 1b, 2a, 2b, 4a, or 4b; and
determining activation or suppression of the Fc receptor CD16a.

25. The method of claim 24 wherein activation is determined by antibody independent cellular cytotoxicity (AICC).

26. The method of claim 24 wherein activation or suppression is determined by measuring luciferase production from a CD16a-luciferase reporter cell line in the presence or absence of an IgG antibody.

27. The method of claim 24 wherein activation is determined by measuring cell production of TNFα, IFNγ, Granzyme B, GM-CSF, or IL1β.

28. A composition comprising Compound 27.

29. A method for manufacturing Compound 27, comprising: reductively aminating Compound 21 in methanol, dichloromethane, and piperidine.

30. A composition comprising Compound 26.

31. A method for manufacturing Compound 26, comprising reductively aminating Compound 21 and isopropylamine in methanol and dichloromethane.

32. A composition comprising Compound 29.

33. A method for manufacturing Compound 29, comprising: oxidizing Compound 21 in the presence of sodium perchlorate, sodium dihydrogen phosphate, and 2-methyl-2-butene in t-butyl alcohol in tetrahydrofuran.

34. A reagent for enhancing in vitro antibody-based assays employing binding of Fc receptor CD16a and an antibody, comprising a compound selected from Compound 1a, 1b, 2a, 2b, 4a, and 4b, or combinations thereof, wherein the compound is not lanosta-8,24-dien-3β-ol or 9β-19-cyclo-24-lanosten-3β-ol.

35. A kit comprising a reagent for enhancing in vitro antibody-based assays employing binding of Fc receptor CD16a and an antibody, comprising:
a compound selected from Compound 1a, 1b, 2a, 2b, 4a, and 4b, or combinations thereof; and
a cell free preparation of Fc receptor CD16a.

36. The kit of claim 35 comprising 3,3′,5,5′-Tetramethylbenzidine (TMB).

37. The method of claim 1 wherein the compound is Compound 27.

38. The method of claim 9 wherein the compound is Compound 27.


SEQUENCE LISTING (5 sequences)

SEQ ID NO: 1 - protein, 211 aa, Homo sapiens
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGGLNDIFEAQKIEWHEHHHHHH

SEQ ID NO: 2 - protein, 211 aa, Homo sapiens
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGGLNDIFEAQKIEWHEHHHHHH

SEQ ID NO: 3 - protein, 300 aa, Homo sapiens
QAAAPPKAVLKLEPPWINVLQEDSVTLTCQGARSPESDSIQWFHNGNLIPTHTQPSYRFKANNNSGEYTCQTGQTSLSDPVHLTVLSEWLVLQTPHLEFQEGETIMLRCHSWKDKPLVKVTFFQNGKSKFSHLDPTFSIPQANHSHSGDYHCTGNIGYTLFSSKPVTITVQVPSMGSSSPMGIIVAVVIATAVAAVAAVVALIYCRKKRISANSTDPVKAAQFEPPGRQMIAIRKRQLEETNNDYETADGGYMTLNPRADDDKNIYLTLPPNDHVNSNNGLNDIFEAQKIEWHEHHHHHH

SEQ ID NO: 4 - protein, 196 aa, Mus musculus
ALPKAVVKLDPPWIQVLKEDMVTLMCEGTHNPGNSSTQWFHNGRSIRSQVQASYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTGLNDIFEAQKIEWHEHHHHHH

SEQ ID NO: 5 - protein, 204 aa, Mus musculus
GLQKAVVNLDPKWVRVLEEDSVTLRCQGTFSPEDNSIKWFHNESLIPHQDANYVIQSARVKDSGMYRCQTALSTISDPVQLEVHMGWLLLQTTKWLFQEGDPIHLRCHSWQNRPVRKVTYLQNGKGKKYFHENSELLIPKATHNDSGSYFCRGLIGHNNKSSASFRISLGDPGSPSMFPPWHQGLNDIFEAQKIEWHEHHHHHH